(19) United States
(12) Patent Application Publication
(10) Pub. No.: US 2021/0009672 A1
(43) Pub. Date: Jan. 14, 2021

(54) METHODS OF TREATING OR PREVENTING LIVER FIBROSIS WITH INHIBITION OF ACTIVINS A & B

(71) Applicant: The Trustees of Indiana University, Indianapolis, IN (US)

(72) Inventors: Guoli Dai, Indianapolis, IN (US); Yan Wang, Carmel, IN (US); Benjamin C. Yaden, Greenwood, IN (US)

(21) Appl. No.: 16/923,282

(22) Filed: Jul. 8, 2020

Related U.S. Application Data
(60) Provisional application No. 62/871,255, filed on Jul. 8, 2019.

(51) Int. Cl. C07K 16/22; A61P 1/16

(52) U.S. Cl. CPC C07K 16/22; C07K 2317/31; A61P 1/16

(57) ABSTRACT

The invention relates to methods to modulate liver fibrosis in a subject by administering an activin inhibitor to the subject. In embodiments of the invention, the activin inhibitor can be an activin A antibody or fragment thereof, an activin B antibody or fragment thereof or both an activin A and activin B antibody or fragments thereof. The methods described can be used to treat or modulate liver fibrosis caused by several diseases or disorders including: autoimmune hepatitis, biliary obstruction, iron overload nonalcoholic fatty liver disease, which includes nonalcoholic fatty liver and nonalcoholic steatohepatitis, viral hepatitis B, hepatitis C, alcoholic liver disease or the long-term consumption of alcohol.

### FIELD OF THE INVENTION

The invention relates to methods to modulate liver fibrosis in a subject by administering an activin inhibitor to the subject. In embodiments of the invention, the activin inhibitor can be an activin A antibody or fragment thereof, an activin B antibody or fragment thereof or both an activin A and activin B antibody or fragments thereof. The methods described can be used to treat or modulate liver fibrosis caused by several diseases or disorders including: autoimmune hepatitis, biliary obstruction, iron overload nonalcoholic fatty liver disease, which includes nonalcoholic fatty liver and nonalcoholic steatohepatitis, viral hepatitis B, hepatitis C, alcoholic liver disease or long-term alcohol consumption.

### BACKGROUND OF THE INVENTION

Liver fibrosis occurs typically following injury or inflammation in the liver. The liver's cells stimulate wound healing. During this wound healing, excess proteins such as collagen and glycoproteins build up in the liver. Eventually, after many instances of repair, the liver cells or hepatocytes can no longer repair themselves. The excess proteins form scar tissue or fibrosis. Several types of liver diseases exist that can cause fibrosis. These include autoimmune hepatitis, biliary obstruction, iron overload nonalcoholic fatty liver disease, which includes nonalcoholic fatty liver (NAFL) and nonalcoholic steatohepatitis (NASH), viral hepatitis B and C (HBV and HCV, respectively), and alcoholic liver disease. It is generally accepted that leading causes of liver fibrosis is i) nonalcoholic fatty liver disease (NAFLD), and ii) alcoholic liver disease (ALD) due to long-term consumption of alcohol.

Although there has been progress in understanding hepatic fibrosis, the development of interventions designed to impede or reverse hepatic fibrosis, while available, have lagged behind. Yoon et al., “Antifibrotic Therapies: Where Are We Now?,” Seminar Liver Dis 2016; 36: p. 87. Perhaps the greatest change in the field of antifibrotic therapy has been the intense focus on NASH as a therapeutic target, reflecting the growing appreciation of this disease as a public health threat, combined with the realization that with cures for HCV in the majority of patients due to direct acting antiviral therapies, fewer HCV and HBV patients typically need antifibrotic therapies, although in reality cirrhosis due to HCV remains a large unmet need. See Sanyal et al., “Trials and tribulations in drug development for nonalcoholic steatohepatitis,” Clin Gastroenterol Hepatol 2014; 12: p. 2104; Udompap et al., “Increasing prevalence of cirrhosis among U.S. adults aware or unaware of their chronic hepatitis C virus infection,” J Hepatol 2016; 64: p. 1027.

Typically, dense cirrhosis with nodule formation, portal hypertension, and early liver failure is generally considered irreversible, but less advanced lesions can show remarkable reversibility when the underlying cause of the liver injury is controlled and possibly by other therapeutic interventions. In studies of patients with HBV, HCV, and NASH, up to 70 percent of patients had reversal of cirrhosis following successful antiviral therapies or bariatric surgery, respectively. See Marcellin et al., “Regression of cirrhosis during treatment with tenofovir disoproxil fumarate for chronic hepatitis B: a 5-year open-label follow-up study,” Lancet 2013; 381: p. 468; D'Ambrosio et al., “A morphometric and immunohistochemical study to assess the benefit of a sustained virological response in hepatitis C virus patients with cirrhosis,” Hepatology 2012; 56: p. 532; Lassailly et al., “Bariatric Surgery Reduces Features of Nonalcoholic Steatohepatitis in Morbidly Obese Patients,” Gastroenterology 2015; 149: p. 379.

The initiation and progression of liver fibrosis are driven by complicated cellular and molecularly mechanisms. Damaged hepatocytes and cytokines released from the inflammatory cells such as Kupffer cells can directly or indirectly activate hepatic stellate cells (HSCs) into myofibroblasts, leading to the accumulation of collagen I, III and other extracellular matrix (ECM) components and thus, liver fibrosis.

Activins are dimers formed by four inhibin subunits-inhibin inhibin βB, inhibin PC, and inhibin PE in mammals. Widely expressed inhibin PA and inhibin βB genes are essential for inducing mesoderm formation during development and follicle stimulating hormone production. The inhibin PC and inhibin PE are expressed predominantly in the liver and are dispensable during development and for maintaining adult homeostasis. Activins A, B, AB, C, and E represent homo- or heterodimers of inhibin PAPA, βBβB, βAβB, βCβC, and βEβE, respectively. Activins A, B, and AB signal through activin receptors/Smad2/3 pathway, whereas activins C and E may not. Activin A is expressed and secreted by hepatocytes and non-parenchymal cells such as HSCs, cholangiocytes and endothelial cells in liver. Several studies demonstrated that activin A induces the activation of HSCs and macrophages and the apoptosis of hepatocytes in vitro. An in vivo study showed that neutralizing activin A mildly reduced CCl₄-induced acute liver injury in mice. However, whether it is associated with liver fibrosis is still unknown.

As structurally related proteins, activin B shares 63% identity and 87% similarity to activin A. Both ligands bind to the activin receptors II and I, and multiple common AP-1 sites in the promoters of both inhibin PA (subunit of activin A) and inhibin βB (subunit of activin B) have been identified, which suggests that activin B may act similarly to activin A in mediating liver pathogenesis. Hepatocytes constitutively express abundant inhibin βA but relatively low inhibin βB. However, hepatic inhibin βB expression is highly upregulated in CCl₄-induced acute liver injury. Recently, activin B was shown to upregulate hepcidin expression in hepatocytes via Smad1/5/8 signaling in response to several inflammatory insults in mice. These findings suggest that activins have a role in mediating hepatic inflammatory response.

Currently available antifibrotic therapies have been directed against suppressing hepatic inflammation or injury generally rather than subduing fibrosis. However, what is needed are therapies that include, beside removing injurious stimuli, those directed to specifically suppressing the hepatic inflammatory response.

### SUMMARY OF THE INVENTION

The present invention provides methods to modulate liver fibrosis in a subject by suppressing hepatic inflammation caused by a hepatic inflammatory response. The methods of the invention comprise administering an agent that inhibits activin, i.e. an activin inhibitor.

In some embodiments, the activin to be inhibited is activin A. In other embodiments, the activin to be inhibited is activin B. In still other embodiments both activin A and activin B are inhibited.

In yet other embodiments, the activin inhibitor may be an activin A antibody or fragment thereof. In still other embodiments the activin inhibitor may be an activin B antibody or fragment thereof. Yet other embodiments contemplate that the antibody or fragment thereof may be bivalent, inhibiting both activin A and activin B.

In other embodiments, the activin inhibitor may be a composition where the composition may include either an antibody or fragment thereof for activin A or an antibody or fragment thereof for activin B. In other embodiments, the composition comprises both an antibody or fragment thereof for activin A and an antibody or fragment thereof for activin B.

In some embodiments, the activin inhibitor or inhibitors is administered once per day. In other embodiments, the activin inhibitor is administered two or more times daily. In other preferred embodiments, the activin A or the antibody B antibody or fragment thereof may be administered once per week.

In still other embodiments, composition containing the activin inhibitor or inhibitors is administered one time daily. In other embodiments, composition is administered two or more times daily. In other preferred embodiments, the composition may be administered once per week.

In any of the embodiments of the invention, administration of the activin inhibitor or inhibitors or composition containing the same occurs by any conventional means including orally intramuscularly, intraperitoneally or intravenously into the subject.

In any embodiment, the activin inhibitor or inhibitors or composition containing the same are injected at a single site per dose or multiple sites per dose.

In any embodiment of the current invention, all of the materials can be packaged into a kit containing all of the necessary components to carry out the claimed methods.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIGS. 1A-E illustrate that liver and serum activin B increases in the patients with liver fibrosis. FIG. 1A shows the mRNA expression of hepatic inhibin βA and inhibin βB and FIG. 1B shows the proteins of hepatic activin A and B in healthy controls and patients with cirrhosis. FIG. 1C shows the concentrations of serum activin A and B in healthy controls, heavy drinkers without liver diseases, and heavy drinkers with liver disease. FIG. 1D shows that activin A and B proteins in the livers of patients with different stages of NASH (F0, F1, F3, and F4). FIG. 1E shows activin A and B proteins in the serum of patients with different stages of NASH (F0, F1, F3, and F4).

FIGS. 2A-K illustrate that liver and serum activin B increases in mice with acute liver injures and liver fibrosis. FIG. 2A shows that the mRNA expression of hepatic inhibin βB at indicated time points after single CCl₄ or vehicle administration in mice. FIG. 2B shows activin B protein quantified in the livers at 6 and 24 hours after single CCl₄ or vehicle treatment in mice. FIG. 2C shows activin B protein quantified in the serum at 6 and 24 hours after single CCl₄ or vehicle treatment in mice. FIG. 2D shows the mRNA expression of hepatic inhibin βA at indicated time points after single CCl₄ or vehicle administration in mice. FIG. 2E shows activin A protein in the livers at 6 and 24 hours after single CCl₄ or vehicle treatment in mice. FIG. 2F shows activin A protein in the serum at 6 and 24 hours after single CCl₄ or vehicle treatment in mice. Following CCl₄ or vehicle dosed twice per week for 4 weeks in mice, FIG. 2G shows mRNA expression of hepatic inhibin βA and inhibin βB. FIG. 2H shows concentrations of serum activin B protein and FIG. 2I shows inhibin inhibin βB-, and TGFβ1-expressing cells visualized with in situ hybridization on liver sections using mouse inhibin A and inhibin B RNAscope probes and a 2.5 HD Assay-Brown kit. Ten days post-oral alcohol plus binge administration in mice, FIG. 2J shows hepatic inhibin βA and inhibin βB transcript levels and FIG. 2K shows hepatic activin A and activin B protein contents.

FIGS. 3A-H illustrate that activin B antibody, activin A antibody, and combination of them show distinct effects in preventing liver fibrosis induced by CCl₄ in mice. Adult female mice were subjected to CCl₄ or vehicle injection (i.p.) twice per week for 4 weeks. Half an hour before the first CCl₄ injection, mice were treated (s.c.) with IgG (60 mg/kg), activin A antibody (10 mg/kg of activin A antibody+50 mg/kg of IgG), activin B antibody (50 mg/kg of activin B antibody+10 mg/kg of IgG), or combination of activin A and activin B antibodies (10 mg/kg of activin A antibody+50 mg/kg of activin B antibody). Thereafter, antibody treatments were performed once per week. As shown in FIG. 3A, four weeks after the initial CCl₄ injection, ALT in the blood was analyzed. As shown in FIG. 3B, four weeks after the initial CCl₄ injection, AST in the blood was analyzed. As shown in FIG. 3C, four weeks after the initial CCl₄ injection, glucose in the blood was analyzed. As shown in FIG. 3D, four weeks after the initial CCl₄ injection, total bilirubin in the blood was analyzed. Representative liver sections stained with Masson's trichrome are shown in FIG. 3E. Percent Masson's trichrome staining areas were quantified by ImagJ are shown in FIG. 3F. FIG. 3G shows the mRNA expression of hepatic Col1α1 was evaluated by qRT-PCR. FIG. 3H shows transcripts of the genes indicated were quantified by qRT-PCR in liver tissues. Data are expressed as means±S.E.M. (n=10). *, P<0.05 compared to vehicle controls. #, P<0.05, compared to IgG controls.

FIGS. 4A-I illustrate that activin B antibody, activin A antibody, and combination of them display different effects in regressing liver fibrosis induced by CCl₄ in mice. Adult female mice were subjected to CCl₄ or vehicle injection (i.p.) twice per week for 10 weeks. Starting from the seventh week, these mice were treated (s.c.) with IgG (60 mg/kg), activin A antibody (10 mg/kg of activin A antibody+50 mg/kg of IgG), activin B antibody (50 mg/kg of activin B antibody+10 mg/kg of IgG), or combination of activin A and activin B antibodies (10 mg/kg of activin A antibody+50 mg/kg of activin B antibody) weekly. As shown in FIG. 4A, ten weeks after the initial CCl₄ injection, ALT in the blood was analyzed. As shown in FIG. 4B, ten weeks after the initial CCl₄ injection, AST in the blood was analyzed. As shown in FIG. 4C, ten weeks after the initial CCl₄ injection, glucose in the blood was analyzed. As shown in FIG. 4D, ten weeks after the initial CCl₄ injection, total bilirubin in the blood was analyzed. Representative liver sections stained with Masson's trichrome are shown in FIG. 4E. Percent Masson's trichrome staining areas were quantified by ImagJ as shown in FIG. 4F. Representative liver MPO and F4/80 immune-histological staining images are shown in FIG. 4G. FIG. 4H shows quantification of percent positive staining area of MPO. FIG. 4I shows quantification of percent positive staining area of F4/80. Data are presented as means±S.E.M. (n=8). *, P<0.05 compared to vehicle controls. #, P<0.05, compared to IgG controls.

FIGS. 5A-E illustrate that activin B is produced in primary mouse hepatocytes (PMHs) and induces differentiation of these cells. PMHs were isolated from adult male mice and cultured overnight. Subsequently, the cells were treated with vehicle (corn oil), lipopolysaccharide (LPS, 10 μg/ml), or 0.5% CCl₄ for 24 hours. As shown in FIG. 5A, cell viability was analyzed. As shown in FIG. 5B, supernatant ALT and AST were analyzed. As shown in FIG. 5C, supernatant activin A and B proteins were analyzed. FIG. 5D shows cell viability of primary hepatocytes after treatment for 24 hours with 0.5% CCl₄ and co-treatment with IgG, activin A antibody, activin B antibody, or combination of both antibodies (100 ng/ml each). FIG. 5E shows the mRNA levels of the genes indicated were evaluated by qRT-PCR in PMHs treated with activin A and B (100 ng/ml each), or their combination for 24 hours. For all above assays, data are expressed as means±S.E.M. *, P<0.05 vs. vehicle controls.

FIGS. 6A-B illustrate that activin B induces macrophages to express inflammatory cytokines or chemokines. FIG. 6A shows transcripts of the genes indicated were quantified by qRT-PCR in RAW264.7 cells after exposure to activin A (100 ng/ml), activin B (100 ng/ml), or their combination (100 ng/ml each) for 24 hours. FIG. 6B shows the mRNA expression of iNOS was evaluated with qRT-PCR in RAW264.7 cells following vehicle or CXCL1 (100 ng/ml) treatment for 6 or 24 hours. For above quantitative analyses, data are presented as means±S.E.M. *, P<0.05 vs. vehicle controls.

FIGS. 7A-F illustrate that activin B morphologically and molecularly activates HSCs. As shown in FIG. 7A, LX-2 cells were treated with bovine serum albumin (BSA, 100 ng/ml), activin A (100 ng/ml), activin B (100 ng/ml), their combination (100 ng/ml each), or TGFβ1 (5 ng/ml) for 24 hours and then underwent 4′,6-diamidino-2-phenylindole (DAPI) staining. As shown in FIG. 7B, LX-2 cells were treated with activin A (100 ng/ml), activin B (100 ng/ml), or TGFβ1 (5 ng/ml) for 6 hours. Total RNAs were isolated, reverse transcribed to cDNA, and then subjected to microarray analysis using HG-U133 plus 2 chips (n=6). Pie chart shows the numbers of genes commonly or uniquely regulated by the individual ligands. FIG. 7C shows the top ten signaling pathways revealed by Ingenuity Canonical Pathway analysis of the 877 target genes shared by these three ligands. FIG. 7D shows a heat-map of the 20 genes exhibiting the highest magnitudes of upregulation or downregulation in response to these three ligands. FIG. 7E and FIG. 7F show LX-2 cells were treated with vehicle, activin A (100 ng/ml), activin B (100 ng/ml), or their combination (100 ng/ml of each) for 24 hours. The expression of the genes indicated was assessed with qRT-PCR. Data are shown as means of fold changes relative to vehicle controls±S.E.M. *, P<0.05.

FIGS. 8A-B show the results of administering antibodies s.c. weekly in C57b/6 female mice for two weeks. There were five treatment groups: IgG 60 mg/kg, Activin B ab 1 mg/kg+IgG 59 mg/kg, Activin B 10 ab mg/kg+IgG 50 mg/kg, Activin B ab 30 mg/kg+IgG 30 mg/kg, and Activin B ab 60 mg/kg (n=8). In FIG. 8A, liver mass was assessed at two weeks after treatment. In FIG. 8B, body weight was assessed at two weeks after treatment. Data are expressed as means S.E.M. Significance is indicated *P<0.05, treated group versus vehicle group (Dunnett's one-way ANOVA).

FIGS. 9A-D show the results of Smad2/3 Binding Element luciferase assays in SBE transfected HEK 293 cells to determine Activin antibodies specificity. SBE transfected HEK293 cells were co-treated with Activin antibodies plus Activin B (FIG. 9A), Activin AB (FIG. 9B), Activin A (FIG. 9C), or Activin C (FIG. 9D) protein for 24 hours.

FIG. 10 illustrates a scheme of how Activin B and A regulate the initiation and progression of liver fibrosis.

FIGS. 11A-G illustrate that activin B antibody, activin A antibody, and combination of them show distinct effects in preventing liver fibrosis induced by BDL in mice. Experimental groups included (1) sham control; (2) IgG+BDL; (3) Activin A antibody+BDL; (4) Activin B antibody+BDL; and (5) Activin A antibody+Activin B antibody+BDL. The first antibody dosing was performed one day prior to BDL surgery and the second dosing one week after BDL. As shown in FIG. 11A, two weeks after BDL, ALT in the blood was analyzed. As shown in FIG. 11B, two weeks after BDL, AST in the blood was analyzed. As shown in FIG. 11C, two weeks after BDL, glucose in the blood was analyzed. As shown in FIG. 11D, two weeks after the BDL, total bilirubin in the blood was analyzed. Two weeks after BDL, representative liver sections stained with Masson's trichrome are shown in FIG. 11E. Two weeks after BDL, percent Masson's trichrome staining areas were quantified by ImagJ are shown in FIG. 11F. FIG. 11G shows the mRNA expression of hepatic Col1α1 was evaluated by qRT-PCR.

FIGS. 12A-C illustrate that Activin B strongly, but Activin A weekly, modulate local and systemic inflammatory cytokines and pro-fibrotic factors during chronic liver injury. FIG. 12A shows that transcripts of the genes indicated were quantified by qRT-PCR in liver tissues. Activin B or dual antibody treatment inhibited the mRNA expression of hepatic CTGF, TGFβ1, iNOS, CXCL1, CXCR2, IL-01, and IL-6, whereas Activin A antibody treatment only suppressed CTGF Furthermore, in bile duct-ligated mice, Activin B antibody or dual antibody treatment reduced CXCL1, IL-6, and IL-10 protein contents in the livers as well as IL-6, TNFα, and IL-2 protein concentrations in the circulation, to similar or distinct extents, whereas Activin A antibody treatment merely decreased the amount of circulating IL-2 protein in the livers (FIG. 12B) and the serum (FIG. 12C).

### DETAILED DESCRIPTION OF THE INVENTION

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The inventors disclose herein methods to modulate liver fibrosis in a subject by suppressing hepatic inflammation caused by an hepatic inflammatory response. The methods of the invention comprise administering an agent that inhibits activin, i.e. an activin inhibitor. The activin to be inhibited can be activin A, activin B or both activin A and activin B. are inhibited.

An activin inhibitor is preferably an antibody directed against an activin. The activin antibody can also include an antibody fragment or a bivalent antibody or fragment thereof, inhibiting both activin A and activin B.

As described herein, the activin inhibitor may be part of a pharmaceutical composition where the composition may include either an antibody or fragment thereof for activin A or an antibody or fragment thereof for activin B or both an antibody or fragment thereof for activin A and an antibody or fragment thereof for activin B.

The activin inhibitor or inhibitors or a composition therein can be administered once per day, two or more times daily or once per week.

The activin inhibitor or inhibitors or composition containing the same can occur by any conventional means including orally intramuscularly, intraperitoneally or intravenously into the subject. If injected, they can be injected at a single site per dose or multiple sites per dose.

It is contemplated that any of the materials disclosed for the methods herein, can be packaged into a kit containing all of the necessary components to carry out the claimed methods.

Activin Antibodies

The activin antibodies described herein can be made or obtained by any means known in the art. For example, U.S. Pat. No. 10,100,109 to Han et al., discloses anti-activin A binding proteins, including antibodies that are fully human, humanized, and chimeric anti-activin A antibodies that bind human activin A, activin A-binding fragments and derivatives of such antibodies, and activin A-binding polypeptides comprising such fragments, the contents of which are incorporated by reference in its entirety.

It is also contemplated that an antibody can be specifically reactive with an activin B polypeptide may also be used as an antagonist. An anti-activin B antibody herein may be an antibody or fragment thereof that binds to the activin B monomer or to a dimer. In some instances, an activin B antibody or fragment thereof may also show binding to other activins, such as activin A, C or E, as well as heterodimers of any of the foregoing. In each case, a preferred antibody will inhibit the effects of activin B on hepatocytes.

Antibody Terminology

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” and other interchangeable terms for similar binding fragments such as described below.

Native antibodies and native immunoglobulins are usually heterotetrameric glycoproteins of about 150,000 Daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}” or “VH”) followed by a number of constant domains (“C_{H}” or “CH”). Each light chain has a variable domain at one end (“V_{L}” or “VL”) and a constant domain (“C_{L}” or “CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The activin inhibitors as described herein can be a “synthetic polypeptide” derived from a “synthetic polynucleotide” derived from a “synthetic gene,” meaning that the corresponding polynucleotide sequence or portion thereof, or amino acid sequence or portion thereof, is derived, from a sequence that has been designed, or synthesized de novo, or modified, compared to an equivalent naturally-occurring sequence. Synthetic polynucleotides (antibodies or antigen binding fragments) or synthetic genes can be prepared by methods known in the art, including but not limited to, the chemical synthesis of nucleic acid or amino acid sequences. Synthetic genes are typically different from naturally-occurring genes, either at the amino acid, or polynucleotide level, (or both) and are typically located within the context of synthetic expression control sequences. Synthetic gene polynucleotide sequences, may not necessarily encode proteins with different amino acids, compared to the natural gene; for example, they can also encompass synthetic polynucleotide sequences that incorporate different codons but which encode the same amino acid (i.e., the nucleotide changes represent silent mutations at the amino acid level).

With respect to activin A or B antibodies, the term “antigen” refers to the proteins activin A or activin B, respectively or any fragment of the protein molecules thereof.

The terms “antigen-binding portion of an antibody,” “antigen-binding fragment,” “antigen-binding domain,” “antibody fragment” or a “functional fragment of an antibody” are used interchangeably herein to refer to one or more fragments of an antibody that retain the ability to specifically bind to activin A or activin B.

It is contemplated that the activin antibodies may also include “diabodies” which refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. See for example, EP 404,097; WO 93/11161; and Hollinger et al., Proc. Natl. Acad. Sci. USA 90:6444 6448 (1993).

It is contemplated that the activin antibodies may also include “chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin are inserted in place of the murine Fc. See for example, Jones et al., Nature 321: 522-525 (1986); Reichmann et al., Nature 332: 323-329 (1988); and Presta, Curr. Op. Struct. Biol., 2: 593-596 (1992).

It is contemplated that the activin antibodies may also include a “monoclonal antibody” which refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which can include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, monoclonal antibodies can be made by a hybridoma method, recombinant DNA methods, or isolated from phage antibody.

As used herein, “immunoreactive” refers to binding agents, antibodies or fragments thereof that are specific to a sequence of amino acid residues on the activin protein (“binding site” or “epitope”), yet if are cross-reactive to other peptides/proteins, are not toxic at the levels at which they are formulated for administration to human use. The term “binding” refers to a direct association between two molecules, due to, for example, covalent, electrostatic, hydrophobic, and ionic and/or hydrogen-bond interactions under physiological conditions and including interactions such as salt bridges and water bridges and any other conventional binding means. The term “preferentially binds” means that the binding agent binds to the binding site with greater affinity than it binds unrelated amino acid sequences.

As used herein, the term “affinity” refers to the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

“Epitope” refers to that portion of an antigen or other macromolecule capable of forming a binding interaction with the variable region binding pocket of an antibody.

The term “specific” refers to a situation in which an antibody will not show any significant binding to molecules other than the antigen containing the epitope recognized by the antibody. The term is also applicable where, for example, an antigen binding domain is specific for a particular epitope which is carried by a number of antigens, in which case the antibody will be able to bind to the various antigens carrying the epitope. The terms “preferentially binds” or “specifically binds” mean that the antibodies bind to an epitope with greater affinity than it binds unrelated amino acid sequences, and, if cross-reactive to other polypeptides containing the epitope, are not toxic at the levels at which they are formulated for administration to human use.

The term “binding” refers to a direct association between two molecules, due to, for example, covalent, electrostatic, hydrophobic, and ionic and/or hydrogen-bond interactions under physiological conditions and includes interactions such as salt bridges and water bridges, as well as any other conventional means of binding.

Formulations

Formulations provided herein may include “pharmaceutical compositions,” in addition to an activin antibody, activin antibodies or antibody fragments thereof, a pharmaceutically acceptable excipient, carrier, buffer, stabilizer, surfactant or other materials well known to those in the art. Such materials should be non-toxic and should not interfere with the efficacy of the active ingredient(s). The precise nature of the carrier or other material will depend on the route of administration.

It is contemplated that any formulation of activin A or activin B antibodies can include pre-filled syringes containing the formulations, and the use of such formulations useful for treating hepatic inflammation or any disorder therein.

In one aspect, the formulation is stable following preparation, which can be tested according to conventional means. Safe handling and administration of formulations comprising proteins represent significant challenges to pharmaceutical formulators. Proteins possess unique chemical and physical properties that present stability problems: a variety of degradation pathways exist for proteins, implicating both chemical and physical instability. Chemical instability includes deamination, aggregation, clipping of the peptide backbone, and oxidation of methionine residues. Physical instability encompasses many phenomena, including, for example, aggregation.

A “stable” formulation is one in which the protein therein essentially retains its physical stability and/or chemical stability and/or biological activity upon storage. Various analytical techniques for measuring protein stability are available in the art. See for example “Peptide and Protein Drug Delivery,” pp. 247-301, Vincent Lee, ed., Marcel Dekker, Inc., New York, N.Y., Pubs. (1991).

Formulations as described herein may contain a buffering agent such as, for example, histidine, acetate, citrate or phosphate. Buffering agents may be included in an amount of about 5 mM to about 100 mM. In one embodiment, the formulation comprises about 5 mM, about 7.5 mM, about 10 mM, about 12.5 mM, about 15 mM, about 17.5 mM, 20 mM, about 22.5 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, or any integer therein histidine, acetate, citrate or phosphate. As used herein, when referring to buffer concentrations, the term “about” means+/−2% of the indicated value.

Formulations may be prepared for any type of administration known for antibodies as described in more detail below.

Formulations provided herein may further include an acceptable carrier or excipient including any carrier or excipient that is a pharmaceutically acceptable carrier or excipient and which is acceptable for administration to a patient as described in in more detail herein.

In one embodiment, a formulation provided herein is isotonic. Representative isotonic formulations include, but are not limited to, those that are from about 250 to about 350 milliosmolar. In another embodiment, a formulation provided herein is hypertonic. Representative hypertonic formulations include, but are not limited to, those that are from about 351 to about 1000 milliosmolar.

Polyols may be added to a formulation described herein in an amount of up to about 1 M. For example, the formulation may comprise polyol in an amount of about 50 mM, about 75 mM, about 100 mM, about 150 mM, about 200 mM, about 225 mM, about 240 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, about 1 M, or any integer therein. In one embodiment, a formulation provided herein contains polyol in an amount of less than 300 mM and the formulation is made isotonic with a salt in a concentration of from about 100 mM to about 175 mM. For example, the formulation containing polyol in an amount of less than 300 mM is made isotonic with a salt in a concentration of about 130 mM. As used herein, when referring to polyol concentrations, the term “about” means+/−2% of the indicated value. In one aspect, a polyol to be used in the formulations provided herein may be a sugar such as, for example, a non-reducing sugar. Representative examples of non-reducing sugars include, but are not limited to, trehalose and sucrose. For example, a formulation may comprise from about 200 mM to about 300 mM trehalose or sucrose. In one embodiment, a formulation may comprise about 240 mM trehalose or sucrose. Alternatively, the sugar may be sorbitol in an amount (concentration) of from about 200 mM to about 300 mM. In one embodiment, a formulation may comprise about 240 mM sorbitol.

A formulation provided herein may have any acceptable pharmaceutically acceptable pH of about 4.0 to about 7.5.

One would understand that formulations comprising an antibody or antigen-binding fragment, identified by the methods described herein can be prepared for storage by mixing the protein having the desired degree of purity with optional physiologically acceptable carriers, excipients and/or stabilizers in the form of aqueous solutions.

Acceptable carriers are physiologically acceptable to the administered patient and retain the therapeutic properties of the compounds with/in which it is administered. Acceptable carriers and their formulations are and generally described in, for example, Remington′ Pharmaceutical Sciences (18th Edition, ed. A. Gennaro, Mack Publishing Co., Easton, Pa. 1990).

One exemplary carrier is physiological saline. The phrase “pharmaceutically acceptable carrier” as used herein means an acceptable material, composition or vehicle, such as a liquid or solid filler, diluent, excipient, and/or solvent involved in carrying or transporting the subject compounds from the administration site of one organ, or portion of the body, to another organ, or portion of the body. Each carrier is acceptable in the sense of being compatible with the other ingredients of the formulation and not injurious to a subject to whom it is administered. Nor should an acceptable carrier alter the specific activity of the subject compounds.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with administration. Compositions or formulations, therefore, refer to a composition suitable for therapeutic and/or diagnostic use in a subject. Compositions and formulations include an amount of a compound described herein and a pharmaceutically or physiologically acceptable carrier.

Compositions can be formulated to be compatible with a particular route of administration (i.e., systemic or local). Thus, compositions include carriers, diluents, or excipients suitable for administration by various routes.

Compositions can be administered, for example, by injection, including, but not limited to, subcutaneous, intradermal, intravenous, intra-arterial, intraperitoneal, or intramuscular injection. Isotonic agents, for example, sugars, polyalcohols such as manitol, sorbitol, and sodium chloride may be included in the composition. The resulting solutions can be packaged for use as is, or lyophilized; the lyophilized preparation can later be combined with a sterile solution prior to administration. For intravenous, injection, or injection at the site of affliction, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as needed. Sterile injectable solutions can be prepared by incorporating an active ingredient in the required amount in an appropriate solvent with one or a combination of ingredients enumerated above, as required, followed by filtered sterilization.

Any formulation can optionally include one or more surfactants, such as, for example, polysorbate 20 or 80, TWEEN, PLURONIC, F68, or polyethylene glycol (PEG).

When the activin compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available packages.

The phrase “pharmaceutically acceptable” refers to molecular entities and compositions that are physiologically tolerable and do not typically produce an allergic or similar untoward reaction, such as gastric upset, dizziness and the like, when administered to a subject.

The term “unit dose” when used in reference to a therapeutic composition refers to physically distinct units suitable as unitary dosage for subjects, each unit containing a predetermined quantity of active material calculated to produce the desired therapeutic effect in association with the required diluent; i.e., carrier, or vehicle.

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable but are typified by an initial administration followed by repeated doses at one or more hour-intervals by a subsequent injection or other administration. Alternatively, continuous intravenous infusions sufficient to maintain concentrations in the blood are contemplated.

One embodiment contemplates the use of the compositions described herein to make a medicament for treating a condition, disease or disorder described herein. Medicaments can be formulated based on the physical characteristics of the patient/subject needing treatment and can be formulated in single or multiple formulations based on the stage of the condition, disease or disorder. Medicaments can be packaged in a suitable package with appropriate labels for the distribution to hospitals and clinics wherein the label is for the indication of treating a subject having a disease described herein. Medicaments can be packaged as a single or multiple units. Instructions for the dosage and administration of the compositions can be included with the packages as described below.

Also provided herein is a pre-filled syringe suitable for intravenous or intraperitoneal administration, comprising a formulation described herein. Such pre-filled syringes may be packaged and labeled for use for treatment of an angiogenesis-related condition such as any of the conditions described herein. Packages may further include directions for storage and administration. Provided herein is a package containing one or more pre-filled syringes suitable for intravenous or intravitreal administration comprising the formulation of any of the preceding claims.

### Other Definitions

As used herein, “prevention” refers to prophylaxis, prevention of onset of, or symptoms, prevention of or modulation liver fibrosis in a subject by suppressing hepatic inflammation caused by an hepatic inflammatory response. The methods of the invention comprise administering an agent that inhibits activin, i.e. an activin inhibitor. As used herein, “inhibition,” “treatment” and “treating” are used interchangeably.

A “subject” or “patient” (e.g., a mammal such as a human or a non-human animal such as a primate, rodent, cow, horse, pig, sheep, camel, llama, etc.) can be a mammal who exhibits one or more clinical manifestations and/or signs or symptoms of a disease or disorder described herein. In certain situations, a subject may be asymptomatic and yet still have clinical manifestations of the disease or disorder.

Disorders to be Treated

The present invention provides methods to modulate liver fibrosis in a subject by suppressing hepatic inflammation caused by a hepatic inflammatory response. The methods of the invention comprise administering an agent that inhibits activin, i.e. an activin inhibitor. It is contemplated that the methods can be used to treat, prevent or modulate any disorder in which liver cells or hepatocytes can no longer repair themselves, for example in instances in which excess hepatic proteins form scar tissue or fibrosis. Thus, the methods disclosed herein can be used to treat any type of liver diseases exist that can cause fibrosis. These include but are not limited to: autoimmune hepatitis, biliary obstruction, iron overload nonalcoholic fatty liver disease, which includes nonalcoholic fatty liver (NAFL) and nonalcoholic steatohepatitis (NASH), viral hepatitis B and C (HBV and HCV, respectively), and alcoholic liver disease. It is generally accepted that leading causes of liver fibrosis is i) nonalcoholic fatty liver disease (NAFLD), and ii) alcoholic liver disease (ALD) due to long-term consumption of alcohol.

In such methods, the formulation may be administered to a patient one or more times. For example, the formulation may be administered once per day, multiple times per day, once per week, once per month, once bi-monthly, once every two months, once every three months, once every four months, once every 5 months, or once every 6 months. Treatment schedules may be increased or decreased as needed depending upon the response of the patient to the treatment. When multiple doses of the composition of the present invention and/or the combined therapeutic moiety are contemplated, it is understood that doses of each can be empirically determined using known doses and concentrations based on the age, height, weight, health and other physical characteristics of a subject using standards of commercially available products.

It is to be understood that by “administering” is referred to herein as providing one or more formulations to a patient in a manner that results in the formulation being inside the patient's body. Such an administration can be by any route including, without limitation, locally, regionally or systemically by subcutaneous, intravitreal, intradermal, intravenous, intra-arterial, intraperitoneal, or intramuscular administration (e.g., injection).

Actual dosage levels of the active ingredients in the formulations can be varied so as to obtain an amount of the active ingredient that is effective to achieve the desired therapeutic response for a particular patient, formulation, and mode of administration, without being toxic to the patient. The selected dosage level will depend upon a variety of factors including the activity of the particular compound employed, the route of administration, the time of administration, the rate of excretion of the particular compound being employed, the duration of the treatment, other drugs, compounds and/or materials used in combination with the particular formulation employed, the age, sex, weight, condition, general health and prior medical history of the patient being treated, and like factors well known in the medical arts.

Formulations can be administered to a patient by any convenient route such as described above. Regardless of the route of administration selected, the compounds of the present invention, which can be used in a suitable hydrated form, and/or the formulations, are formulated into acceptable dosage forms such as described below or by other conventional methods known to those in the art.

Further reference is made to the following experimental examples.

### EXAMPLES

The following examples are given for the purpose of illustrating various embodiments of the invention and are not meant to limit the present invention in any fashion. The present examples, along with the methods described herein are presently representative of preferred embodiments, are provided only as examples, and are not intended as limitations on the scope of the invention. Changes therein and other uses which are encompassed within the spirit of the invention as defined by the scope of the claims will occur to those skilled in the art.

The following applies to any of the appropriate testing described herein in any of the examples.

Blood Biochemistry

Serum aspartate aminotransferase (AST), alanine aminotransferase (ALT), glucose, and total bilirubin levels were measured with a Hitachi Modular Analyzer (Roche Diagnostics, Indianapolis, Ind.).

Histology and Immunohistochemistry

Formalin-fixed and paraffin-embedded liver sections were subjected to a standard procedure of immunohistochemistry with primary antibodies against F4/80 (eBioscience, San Diego, Calif.) and myeloperoxidase (MPO, R&D System, Minneapolis, Minn.). The liver sections were additionally subjected to Masson's trichrome staining. Images were acquired using digital slide scanning (Aperio Technologies, Vista, Calif.) and analyzed the stained area percentage by ImageJ (National Institutes of Health, NIH, USA).

ELISA of Activin A and Activin B

Activin A and activin B proteins in liver tissue, serum, or cell culture supernatants were quantified by ELISA methods (Activin A ELISA kit, Sigma, St. Louis, Mo.; Activin B ELISA kit, Ansh labs, Webster, Tex.) according to the protocols provided by the manufacturers.

Statistical Analysis

Statistical significance, P<0.05, was determined by Dunnett's tests, or a two-tailed unpaired Student's t-test to compare the differences between experimental and control groups. The data were expressed as means±S.E.M. GraphPad Prism Software was used for data analysis and figure preparation.

### Example 1

Levels of Hepatic and Circulating Activin B are Significantly Increased in Patients with Liver Fibrosis.

Levels of mRNA and protein expression of both activin A and activin B were examined in normal patients (controls) and from those with different stages (F0-F4) of NASH. Serum levels of activins were measured in patients with NASH (n=44), heavy drinkers without liver disease (n=36), and those with alcoholic cirrhosis (n=15) compared to normal controls (n=16). Normal liver samples from healthy volunteers (n=5) or from patients with advanced fibrosis or established cirrhosis secondary to non-alcoholic steatohepatitis (NASH) (n=8). Liver samples were collected from the patients with advanced fibrosis or established cirrhosis during their liver transplantation procedure. Demographic data, cirrhosis etiology, and other relevant information such as medication, alcohol use, and smoking history were also obtained. NASH was staged based on the severity of scarring or fibrosis: F0, no scarring; F1, minimal scarring; F2, significant fibrosis; F3, severe fibrosis; and F4, cirrhosis or advanced scarring. See Pavlov et al., “Transient elastography for diagnosis of stages of hepatic fibrosis and cirrhosis in people with alcoholic liver disease,” Cochrane Database Syst Rev 2015; 1:CD010542. Heavy drinkers were defined as those consuming greater than 15 drinks per week for males and 8 drinks per week for females. Blood samples were harvested from healthy controls (n=16), heavy alcohol drinkers without liver disease (n=36), and heavy alcohol drinkers with liver disease (n=15). Liver tissue samples were snap frozen in liquid nitrogen. Serum and frozen liver samples were stored at −80° C. until use.

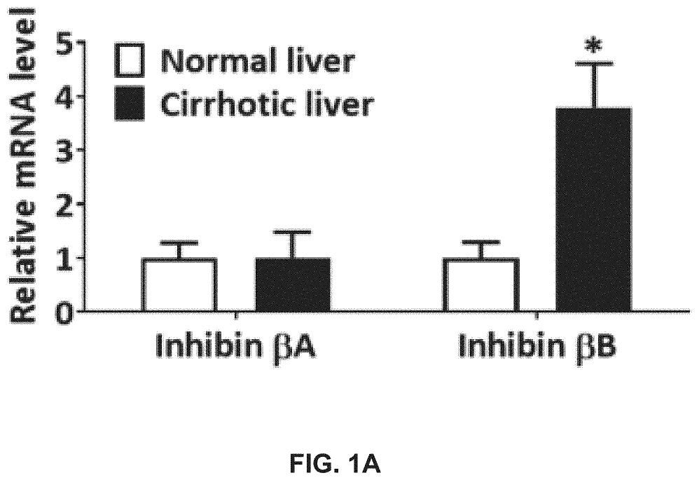

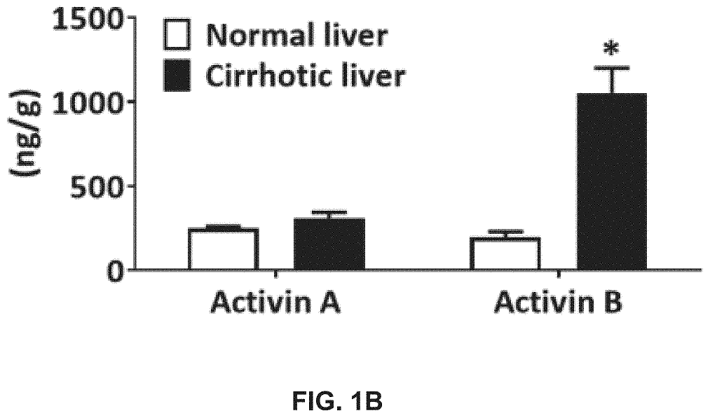

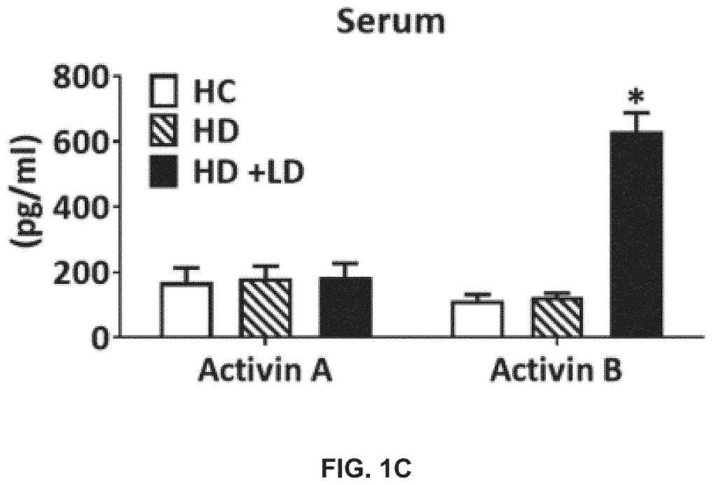

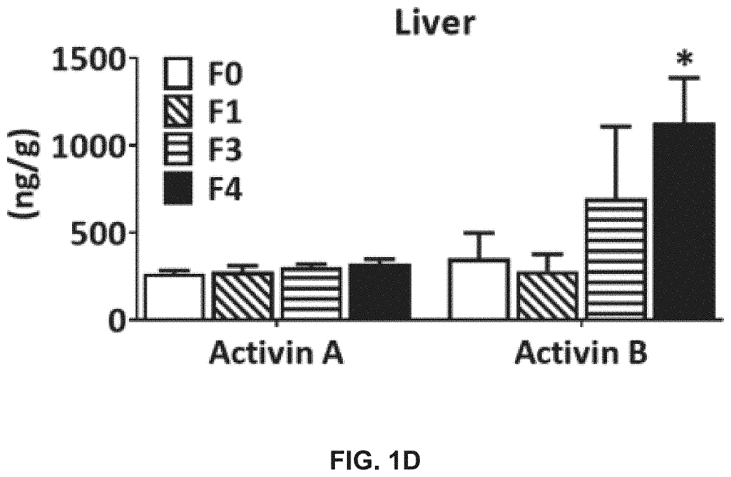

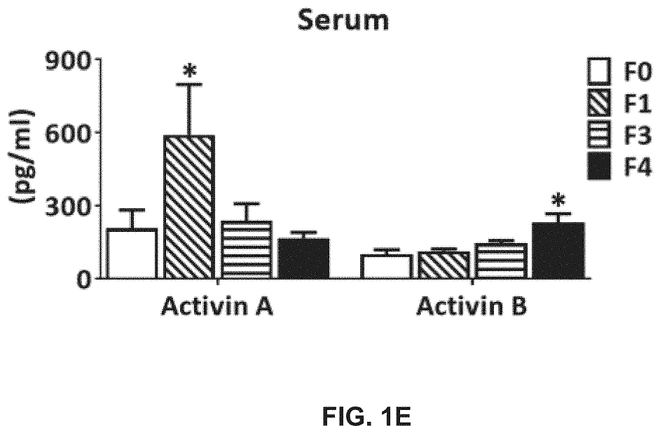

FIGS. 1A-E illustrate that liver and serum activin B increases in the patients with liver fibrosis. FIG. 1A shows the mRNA expression of hepatic inhibin βA and inhibin βB (the subunits of activin A and B, respectively). FIG. 1B shows the proteins of hepatic activin A and B in patients with cirrhosis (n=8) and healthy controls (n=5) were analyzed by qRT-PCR and ELISA respectively. FIG. 1C shows the concentrations of serum activin A and B proteins were determined with ELISA in healthy controls (HC, n=16), heavy drinkers without liver diseases (HD, n=36), and heavy drinker with liver disease (HD+LD, n=15). FIG. 1D shows Activin A and B proteins were evaluated by ELISA in the livers of patients with different stages of NASH (F0: n=4, F1: n=6, F3: n=5, and F4: n=6). FIG. 1E shows Activin A and B proteins were evaluated by ELISA in the serum of patients with different stages of NASH (F0: n=4, F1: n=6, F3: n=5, and F4: n=6). For all above assays in this figure, data are expressed as means±S.E.M. *, P<0.05 compared to healthy controls or F0 group. Note that inhibin βA and βB represent the subunit of activin A and B respectively.

The results demonstrated that the levels of hepatic and circulating activin B are significantly increased in patients with liver fibrosis. It was also determined whether activin B and A are clinically relevant to different etiologies of liver fibrosis. Regarding the mRNA expression, inhibin βA represents activin A and inhibin βB symbolizes activin B as activin A and activin B are the homodimers of inhibin βA and inhibin βB respectively. In patients with advanced liver fibrosis or cirrhosis, hepatic activin B mRNA and protein exhibited marked increases relative to healthy controls (FIGS. 1A and 1B). Circulating activin B did not increase in excessive alcohol users without liver disease but elevated more than five-fold in patients with alcoholic cirrhosis (FIG. 1C). In NASH patients, the hepatic and serum levels of activin B significantly increased only in those with F4 fibrosis compared to F0 and F1 group (FIGS. 1D and 1E). In addition, it was found that the serum level of activin A markedly increased in those with F1 fibrosis (FIG. 1E). Taken together, the expression of activin B is correlated with advanced fibrosis/cirrhosis, irrespectively of underlying disease etiologies.

### Example 2

Levels of Hepatic and Circulating Activin B are Significantly Elevated in a Mouse Model of Liver Fibrosis.

To further investigate the expression pattern and cellular sources of activin B and A in liver injury, acute and chronic liver injury studies in mice were performed. All mouse experiments were performed with the approval of Institutional Animal Care and Use Committee of Eli Lilly and Company and Indiana University-Purdue University Indianapolis. Several mouse models were used for our study. For acute liver injury models, C57BL/6 female mice at the age of 10-12 weeks (Envigo, Indianapolis, Ind.) received a single intraperitoneal administration of CCl₄ (Sigma Aldrich, St. Louis, Mo.) (1:10 dilution in corn oil, 10 ml/kg) for 1.5 hour, 3 hour, 6 hour, 24 hour, and 3 day. For liver fibrosis models, mice were intraperitoneally injected of CCl₄ twice a week for 4 weeks or 10 weeks. See Lee et al., “Fusion protein of retinol-binding protein and albumin domain III reduces liver fibrosis. EMBO Mol Med 2015; 7(6): pp. 819-30; Knockaert et al., “Carbon tetrachloride-mediated lipid peroxidation induces early mitochondrial alterations in mouse liver,” Lab Invest 2012; 92(3): pp. 396-410. For ALD model, ethanol oral feeding lasted for 10 days plus binge as described previously. See Lamas-Paz et al., “Alcoholic liver disease: Utility of animal models,” World J Gastroenterol 2018; 24(45): pp. 5063-75.

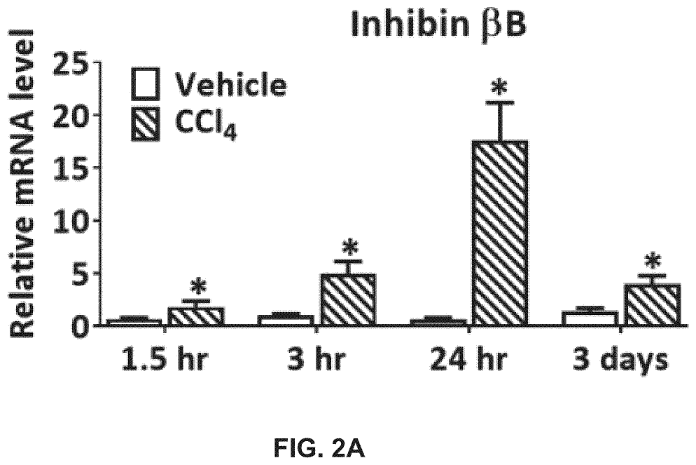

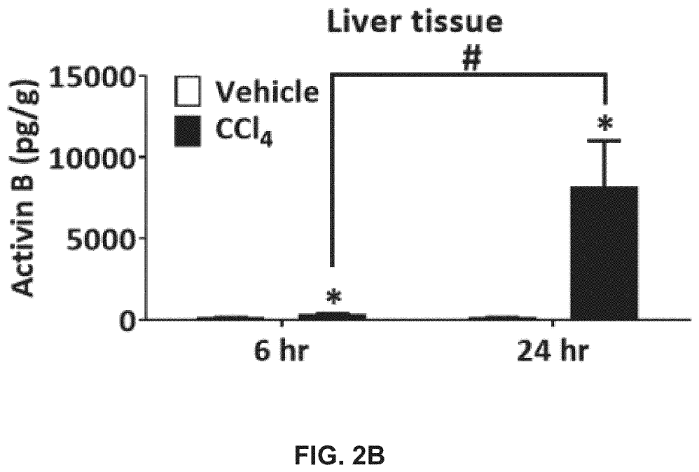

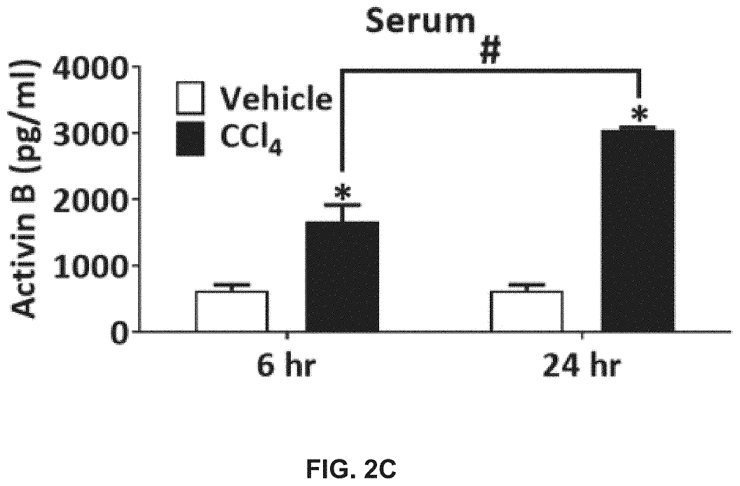

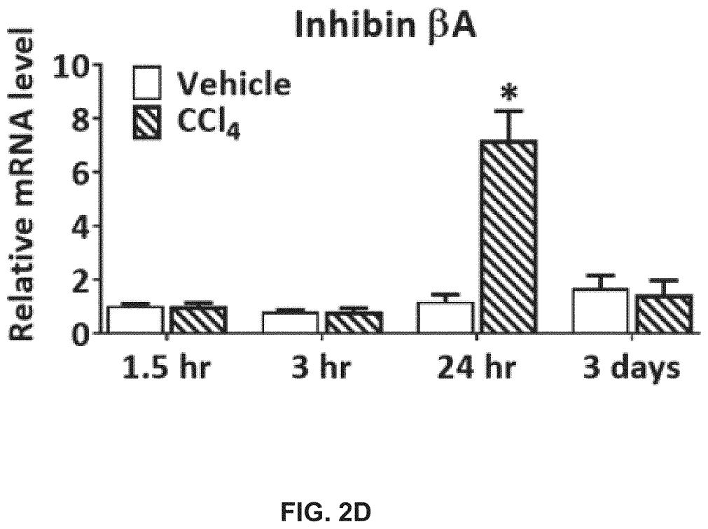

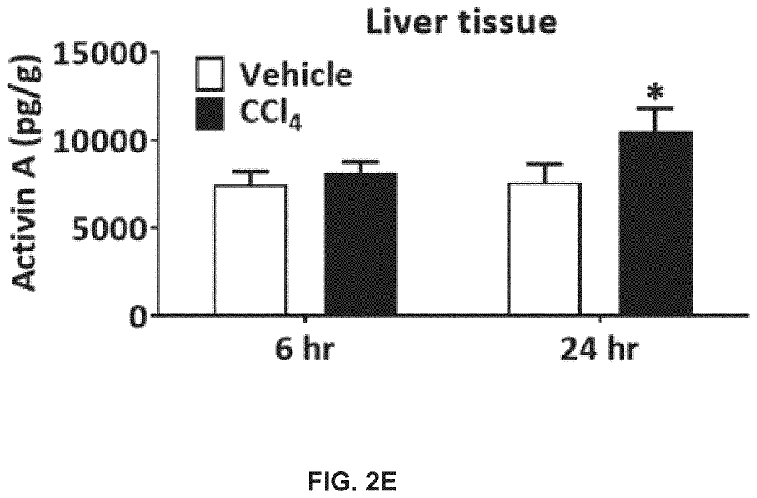

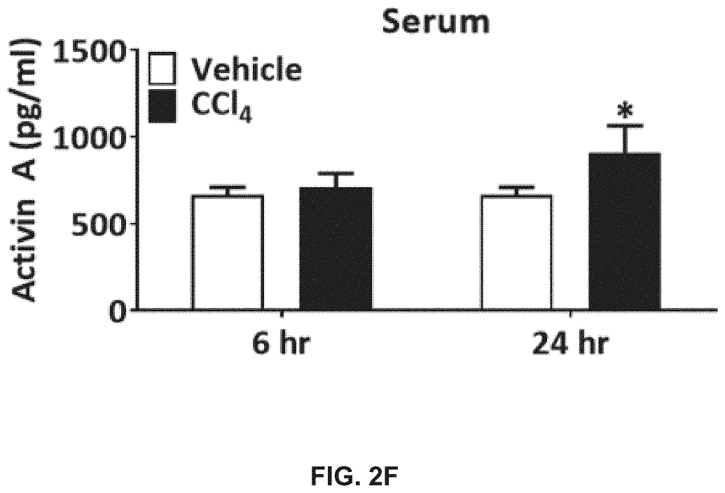

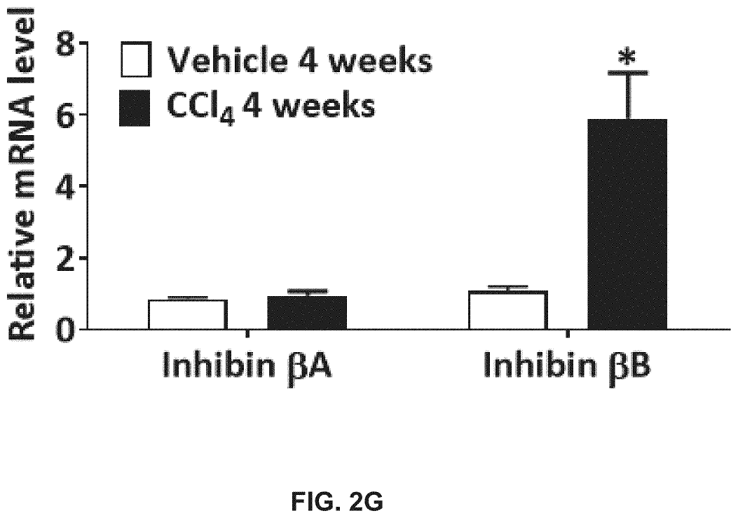

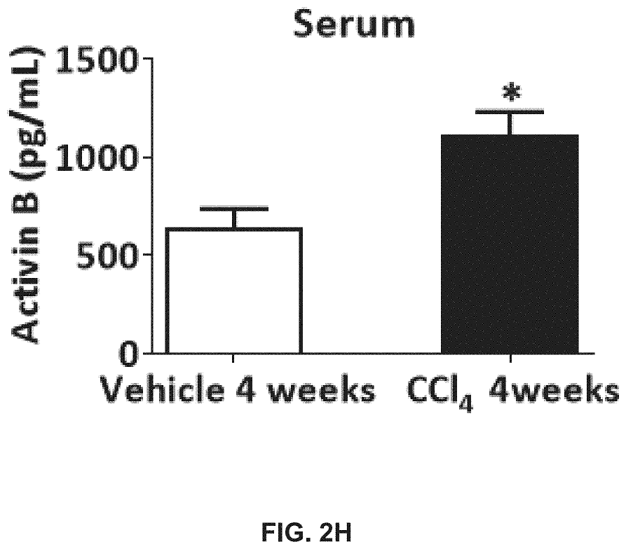

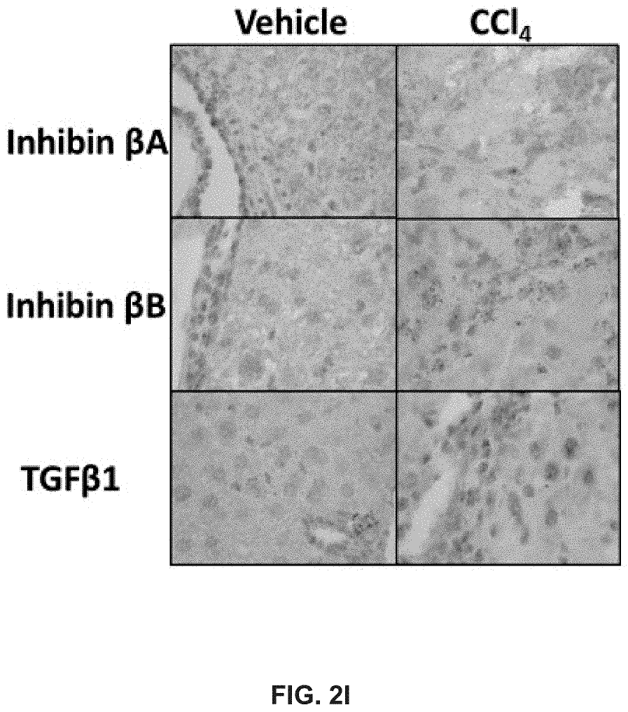

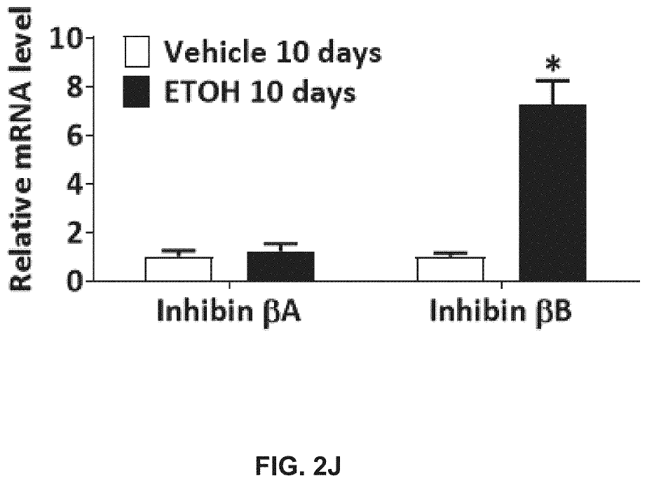

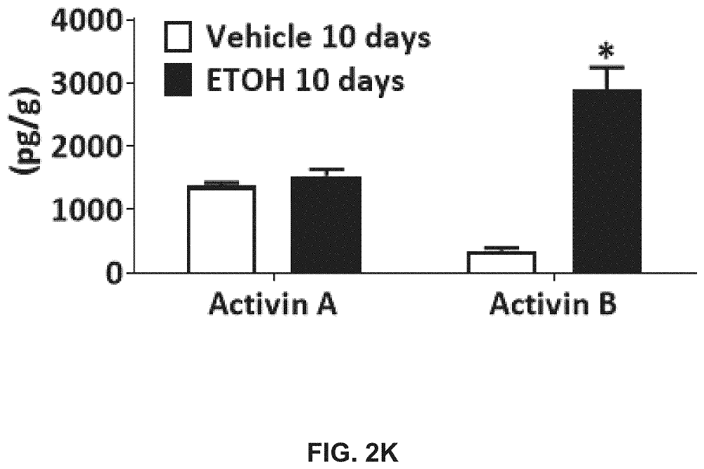

FIGS. 2A-K illustrate that liver and serum activin B increases in mice with acute liver injures and liver fibrosis. FIG. 2A shows that the mRNA expression of hepatic inhibin βB was analyzed by qRT-PCR at indicated time points after single CCl₄ or vehicle administration in mice (n=6). FIG. 2B shows that activin B protein was quantified with ELISA in the livers at 6 and 24 hours after single CCl₄ or vehicle treatment in mice (n=8). FIG. 2C shows that activin B protein was quantified with ELISA in the serum at 6 and 24 hours after single CCl₄ or vehicle treatment in mice (n=8). FIG. 2D shows that the mRNA expression of hepatic inhibin βA was analyzed by qRT-PCR at indicated time points after single CCl₄ or vehicle administration in mice (n=6). FIG. 2E shows that activin A protein was quantified with ELISA in the livers at 6 and 24 hours after single CCl₄ or vehicle treatment in mice (n=8). FIG. 2F shows that activin A protein was quantified with ELISA in the serum at 6 and 24 hours after single CCl₄ or vehicle treatment in mice (n=8). Following CCl₄ or vehicle was dosed twice per week for 4 weeks in mice, FIG. 2G shows that mRNA expression of hepatic inhibin βA and inhibin βB was assessed by qRT-PCR (n=10); FIG. 2H shows that concentrations of serum activin B protein were quantified with ELISA (n=10); and FIG. 2I shows that inhibin inhibin βB-, and TGFβ1-expressing cells were visualized with in situ hybridization on liver sections using mouse inhibin A and inhibin B RNAscope probes and a 2.5 HD Assay-Brown kit. Ten days post-oral alcohol plus binge administration in mice, FIG. 2J shows that hepatic inhibin βA and inhibin βB transcript levels were determined by qRT-PCR (n=7), and FIG. 2K shows that hepatic activin A and activin B protein contents were quantified with ELISA (n=7). For all above quantitative assays, data are expressed as means±S.E.M. *, P<0.05 relative to vehicle controls.

The results demonstrated that the levels of hepatic and circulating activin B are significantly elevated in mouse model of liver fibrosis. Acute and chronic liver injury studies in mice were performed. In an acute liver injury model generated by a single administration of CCl₄, significantly upregulated hepatic inhibin βB mRNA expression was found up to 3 days post injection (FIG. 2A), concomitant with the increase in hepatic activin B protein concentration (FIG. 2B). Additionally, it was also observed the increase in serum activin B protein at 6 and 24 hours post injection (FIG. 2C). In contrast to activin B, it was found that the increases in hepatic mRNA expression, hepatic protein concentration, and serum level of activin A only at 24 hours post CCl₄ injection (FIGS. 2D and 2F). A mouse liver fibrosis model of CCl₄ injection was used for 4 weeks and ALD model of chronic alcohol plus binge to determine the levels of activin B in hepatic fibrogenesis and chronic liver injury. In the CCl₄ model, it was observed that the increases in mRNA expression and serum level only for activin B, but not activin A (FIGS. 2G and 2H). Similar findings were found in mice fed with chronic alcohol plus binge (FIGS. 2J and 2K). The cellular sources of activin B were revealed by in situ hybridization. Activin B and A were mainly transcribed in hepatocytes and biliary epithelial cells in vehicle-controlled livers and additionally in fibrogenic cells in the fibrotic livers (FIG. 2I; discussed in Example 5). Collectively, it was demonstrated that, irrespective of liver injury types, activin B is persistently associated with liver disease progression from acute phase to chronic phase, whereas activin A is transiently relevant to the acute phase. Moreover, the association of activin B with liver fibrosis is highly conserved between humans and mice.

### Example 3

Neutralization of Activin B Prevents CCl₄-Induced Liver Fibrosis.

The studies in humans and mice illustrated in Examples 1 and 2 strongly suggested that activin B and A differently participate in the regulation of liver fibrosis progression, prompting an examination of this hypothesis. Global gene knockouts of these two widely produced activin ligands cause developmental defects, reproductive failure, or postnatal death in mice. Neutralizing antibodies were used to systemically inactivate these two proteins and subsequently examine their effects on the initiation of CCl₄-induced liver fibrosis. There were five treatment groups: (1) vehicle; (2) IgG+CCl₄; (3) activin A antibody+CCl₄; (4) activin B antibody+CCl₄; and (5) combination of both antibodies+CCl₄. In the initial association studies, time windows were found during which both activin A and B were induced in the acute phase of liver injuries (see Example 2 and FIGS. 2A-K). This co-induction suggested a possible spatiotemporal coordination between the two activin ligands, warranting combination antibody treatment in this study.

Choice of Antibody Dose

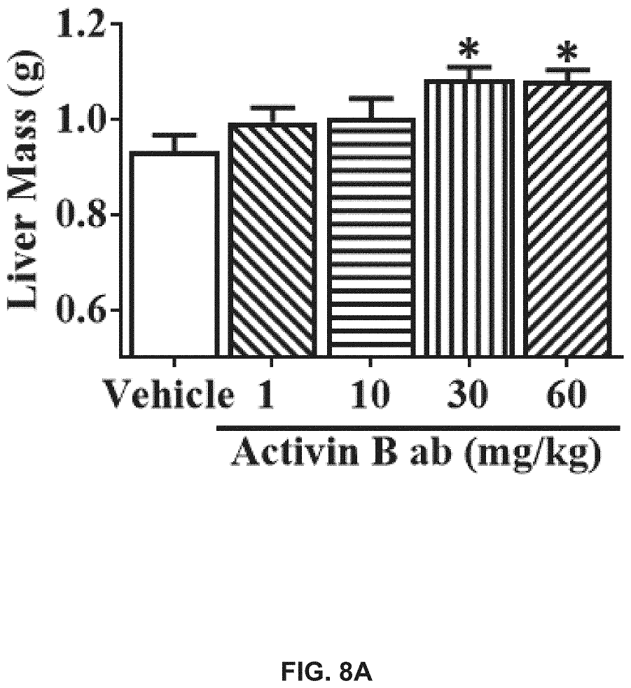

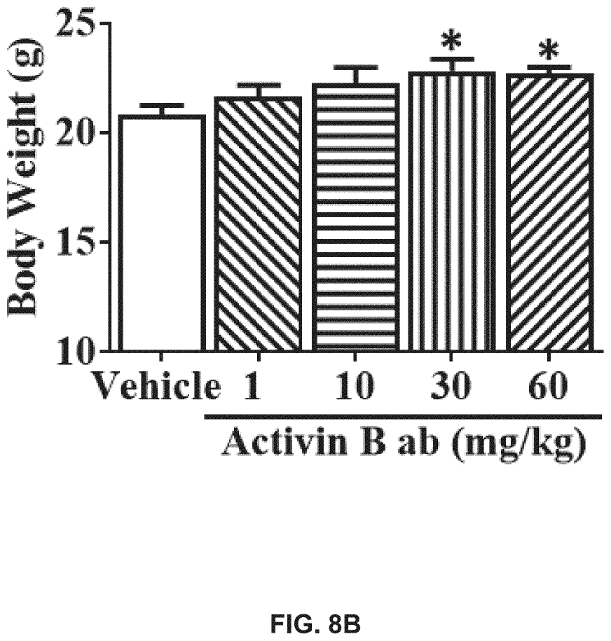

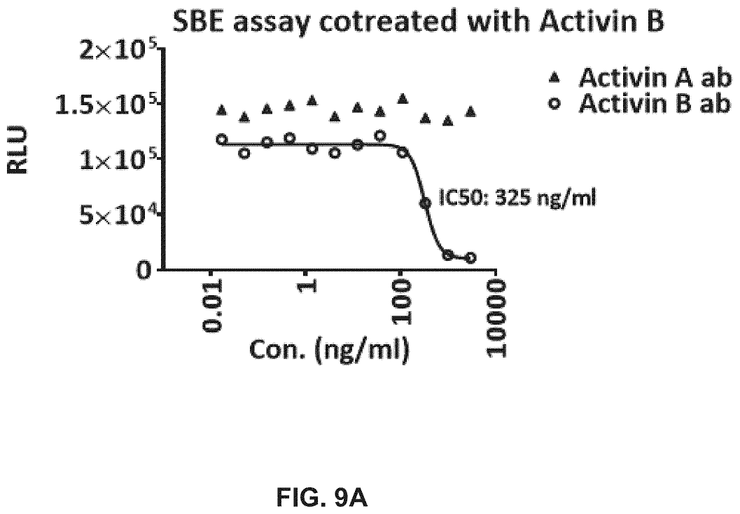

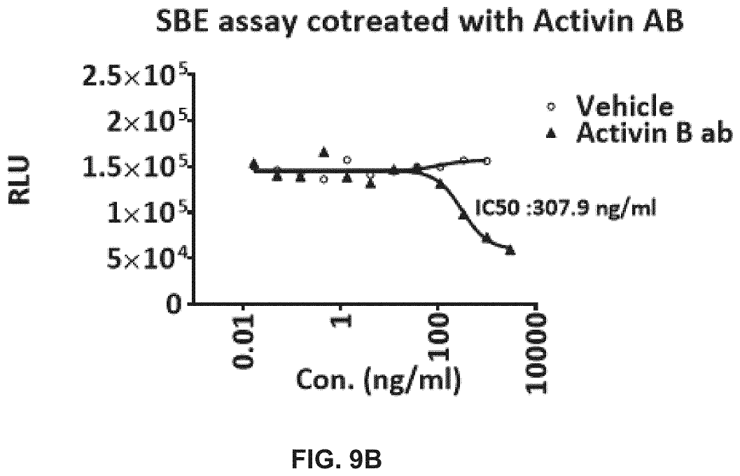

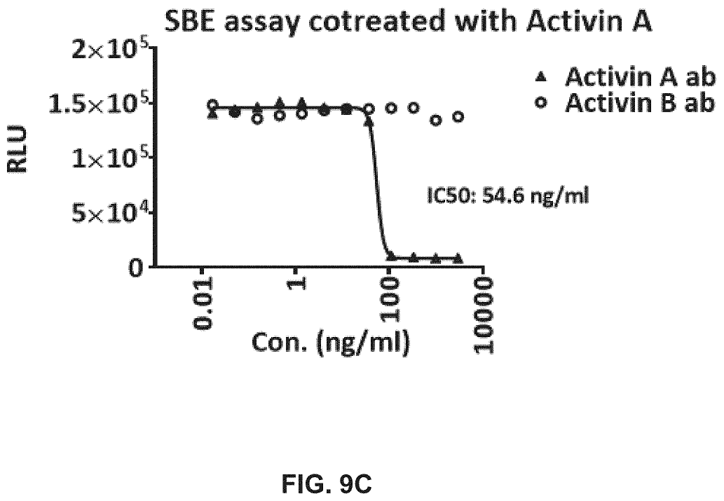

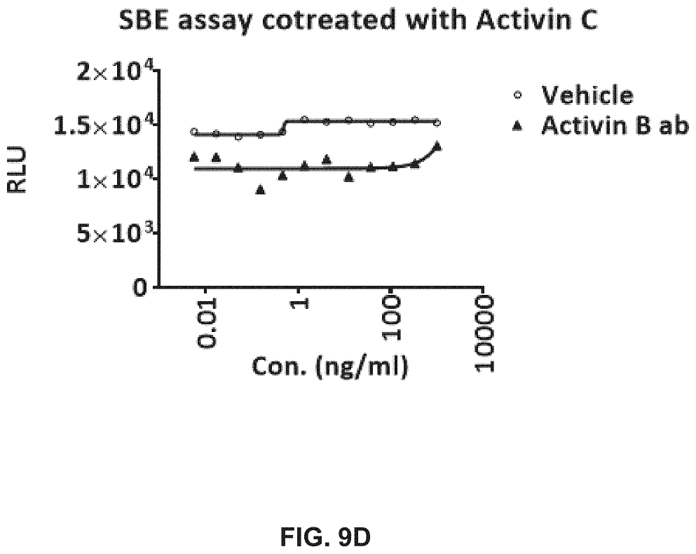

Antibodies were initially dosed half an hour before the first CCl₄ injection and were dosed weekly thereafter. A dosage of 10 mg/kg of activin A antibody weekly was used because a previous study demonstrated the greatest efficacy of this regimen in regressing degeneration of injured skeletal muscle in mice. A dosage of 50 mg/kg was administered as the maximal efficacy dose of activin B antibody once per week because its IC₅₀ was found to be five-fold higher than that of activin A antibody as determined by a Smad2/3 binding element promoter luciferase assay (see FIGS. 9A-D). In addition, in a mouse homeostasis study, this regimen of activating B antibody was sufficient to disrupt liver homeostasis, reflected by an increase in liver mass (FIGS. 8A-B). The results of both of these experiments are shown below.

Smad2/3 Binding

HEK293 cells stably expressing the Smad2/3-binding element (SBE)-12-luciferase system (Qiagen) were seeded at 50,000 to 100,000 cells/well/100 μL DMEM/F12 (Invitrogen) containing 10% FBS into a poly-D-lysine-coated 96-well plate. Following at least 16 h of incubation at 37° C., the media was aspirated and replaced with 50 μL of 1% FBS-DMEM/F12. Anti-activin A mAb or anti-activin B mAb were serially diluted (1:2) with 1×PBS, pH 7.4 to produce the following titration range (3000 ng/mL to 23.4 ng/mL). Each concentration was then mixed with an equal volume of 15 ng/mL of activin A or activin B (R&D Systems) and incubated at room temperature for 30 min, after which 100 μL of the mixture was added to individual wells. The Smad reporter (I.E. 100% signal) was induced by either activin A or activin B alone, and negative controls (I.E. 0% background signal) were induced by vehicle alone. Plates were incubated at 37° C. for 20 h, followed by aspiration, and washed once with 1×PBS. Cells in individual wells were subjected to lysis, and luminescence was measured using a GeniosPRO instrument with substrate injection (Luciferase Reporter Gene Assay Kit, Roche). Values shown in the figures are representative of Smad2/3 binding element reporter assay experiments performed in triplicate. Relative luciferase units were measured, and IC50 curves were fitted using GraphPad Prism software (GraphPad Software, Inc.).

FIG. 9 shows the results of Smad2/3 binding element luciferase assays in SBE transfected HEK 293 cells to determine Activin antibodies specificity. SBE transfected HEK293 cells were co-treated with Activin antibodies plus Activin B (FIG. 9A), Activin AB (FIG. 9B), Activin A (FIG. 9C), or Activin C (FIG. 9D) protein for 24 hours.

Liver Mass Study

Activin B antibody was sufficient to disrupt liver homeostasis, reflected by an increase in liver mass. Antibodies were s.c. administered weekly in C57b/6 female mice for two weeks. There were five treatment groups: IgG 60 mg/kg, Activin B ab 1 mg/kg+IgG 59 mg/kg, Activin B 10 ab mg/kg+IgG 50 mg/kg, Activin B ab 30 mg/kg+IgG 30 mg/kg, and Activin B ab 60 mg/kg (n=8). (A) Liver mass and (B) body weight were assessed at two weeks after treatment. Data are expressed as means±S.E.M. Significance is indicated *P≤0.05, treated group versus vehicle group (Dunnett's one-way ANOVA).

FIGS. 8A-B show the results of administering antibodies s.c. weekly in C57b/6 female mice for two weeks. There were five treatment groups: IgG 60 mg/kg, Activin B ab 1 mg/kg+IgG 59 mg/kg, Activin B 10 ab mg/kg+IgG 50 mg/kg, Activin B ab 30 mg/kg+IgG 30 mg/kg, and Activin B ab 60 mg/kg (n=8). In FIG. 8A, liver mass was assessed at two weeks after treatment. In FIG. 8B, body weight was assessed at two weeks after treatment. Data are expressed as means S.E.M. Significance is indicated *P<0.05, treated group versus vehicle group.

Overall Results of Example 3

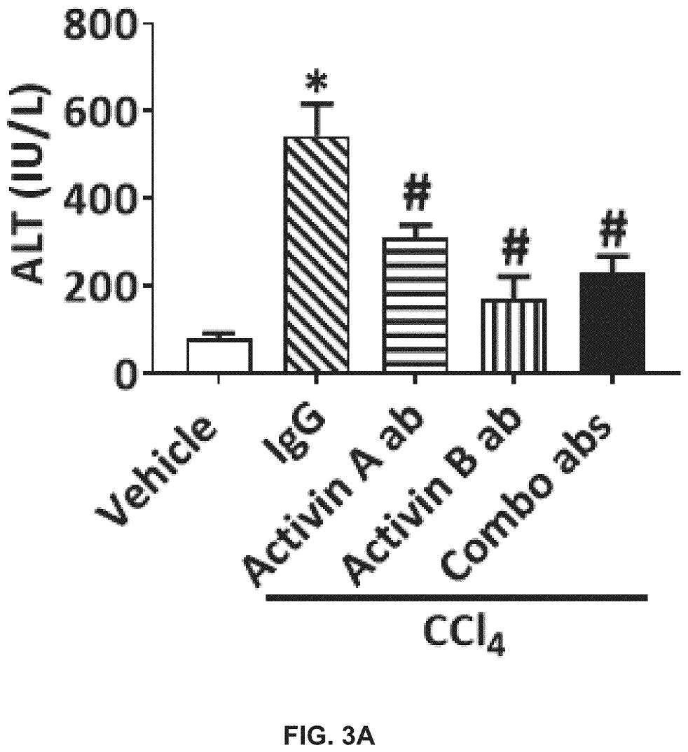

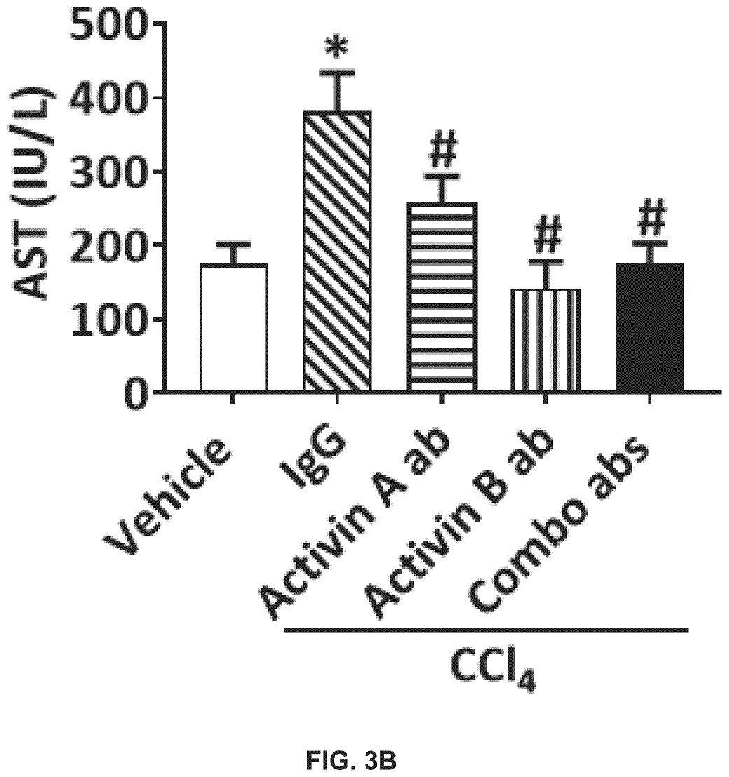

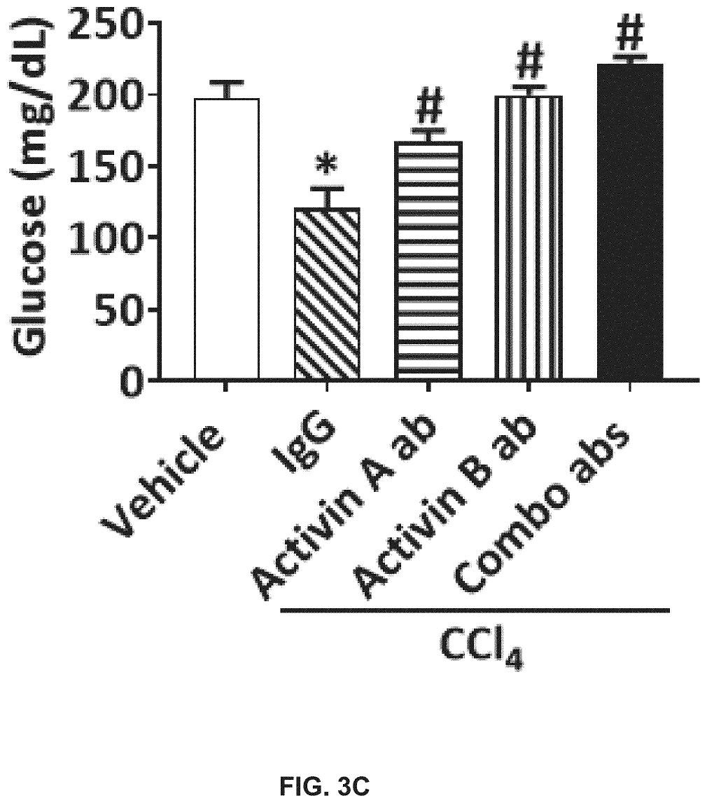

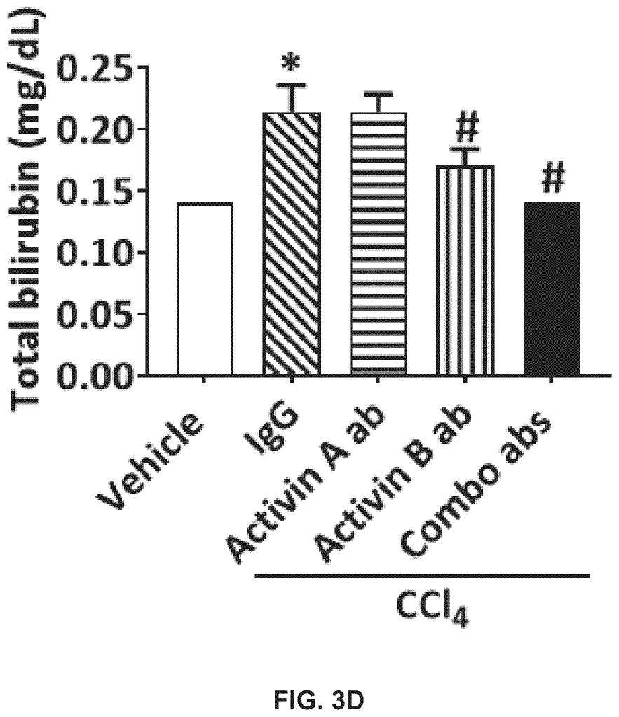

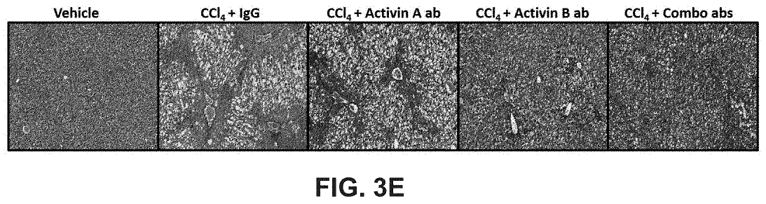

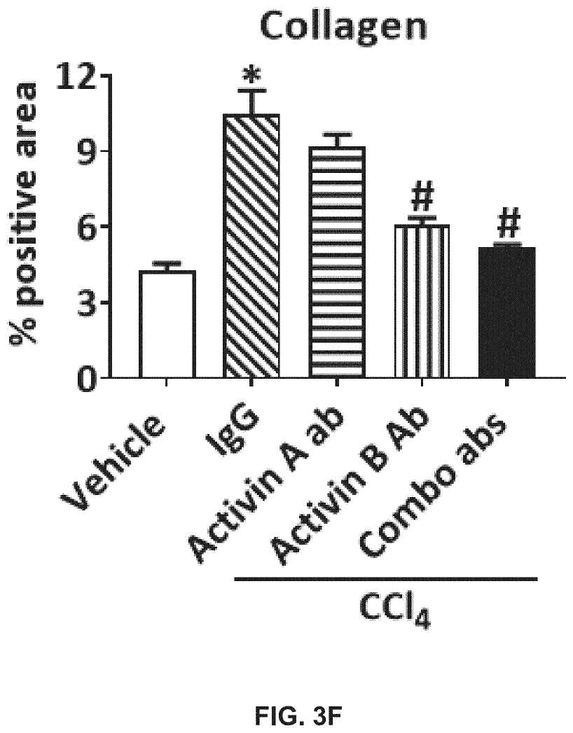

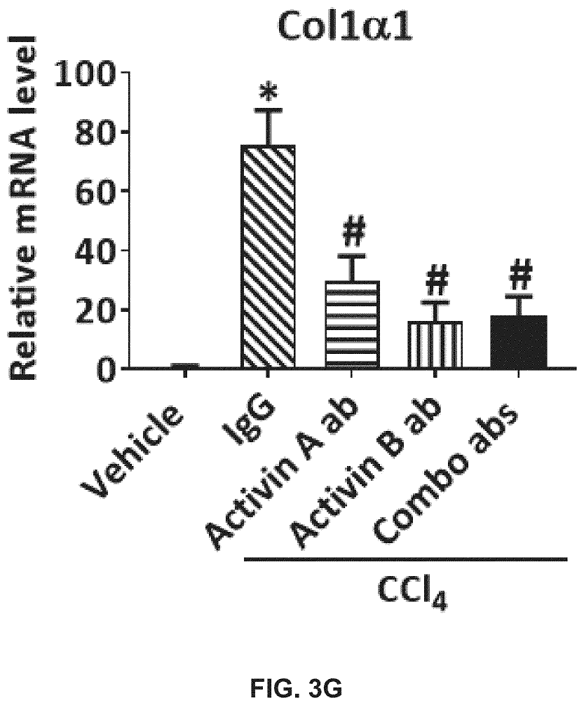

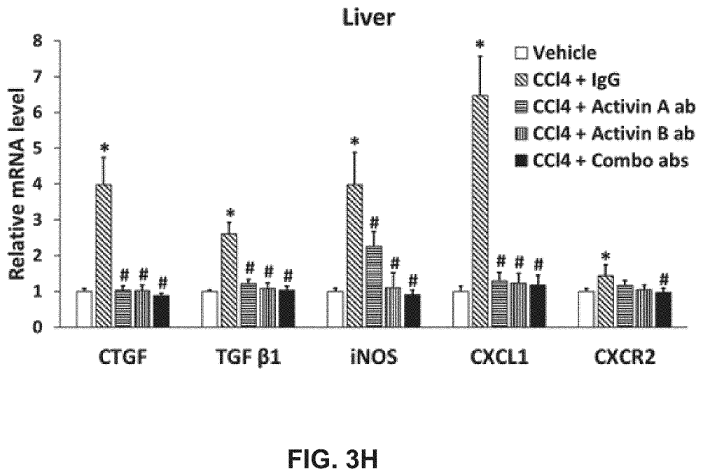

FIGS. 3A-H illustrate that activin B antibody, activin A antibody, and combination of them show distinct effects in preventing liver fibrosis induced by CCl₄ in mice. Adult female mice were subjected to CCl₄ or vehicle injection (i.p.) twice per week for 4 weeks. Half an hour before the first CCl₄ injection, mice were treated (s.c.) with IgG (60 mg/kg), activin A antibody (10 mg/kg of activin A antibody+50 mg/kg of IgG), activin B antibody (50 mg/kg of activin B antibody+10 mg/kg of IgG), or combination of activin A and activin B antibodies (10 mg/kg of activin A antibody+50 mg/kg of activin B antibody). Thereafter, antibody treatments were performed once per week. As shown in FIG. 3A, four weeks after the initial CCl₄ injection, ALT in the blood was analyzed. As shown in FIG. 3B, four weeks after the initial CCl₄ injection, AST in the blood was analyzed. As shown in FIG. 3C, four weeks after the initial CCl₄ injection, glucose in the blood was analyzed. As shown in FIG. 3D, four weeks after the initial CCl₄ injection, total bilirubin in the blood was analyzed. Representative liver sections stained with Masson's trichrome are shown in FIG. 3E. Percent Masson's trichrome staining areas were quantified by ImagJ are shown in FIG. 3F. FIG. 3G shows the mRNA expression of hepatic Col1α1 was evaluated by qRT-PCR. FIG. 3H shows transcripts of the genes indicated were quantified by qRT-PCR in liver tissues. Data are expressed as means±S.E.M. (n=10). *, P<0.05 compared to vehicle controls. #, P<0.05, compared to IgG controls.

As a result, Activin B antibody exerted extensive beneficial effects, including reduced liver injury indicated by serum ALT and AST (FIGS. 3A-B), improved liver function evaluated by serum glucose and total bilirubin level (FIGS. 3C-D), and decreased liver fibrosis analyzed by collagen staining and collagen 1α1 mRNA expression (FIGS. 3E-G). Activin A antibody treatment reduced liver injury and improved liver functions to a lesser extent compared with activin B antibody, but did not decrease total bilirubin and liver fibrosis, although collagen 1α1 mRNA expression was inhibited (FIGS. 3A-G). The dual antibodies showed beneficial effects equivalent to, or in some cases greater than, activin B antibody alone (FIGS. 3A-G). In CCl₄ chronically damaged livers, activin B and A are essential collaborators to induce CXCL1, iNOS, CTGF, and TGFβ1, because neutralizing either one of them prevented or inhibited the upregulation of these genes (FIG. 3H). Together, these data demonstrate that (1) activin B and, to a much lesser extent, activin A mediate the initiation of liver fibrosis; and (2) activin B inhibition or, even better, both activin B and A inhibition prevents liver fibrosis.

### Example 4

Neutralization of Activin B Regresses CCl₄-Induced Liver Fibrosis.

To test the hypothesis that neutralization of activin B regresses CCl₄-induced liver fibrosis, following the same study design as the prevention study, CCl₄ was injected twice per week for ten continuous weeks. Starting at the seventh week when liver fibrosis was fully established, antibodies were dosed weekly for the remaining four weeks.

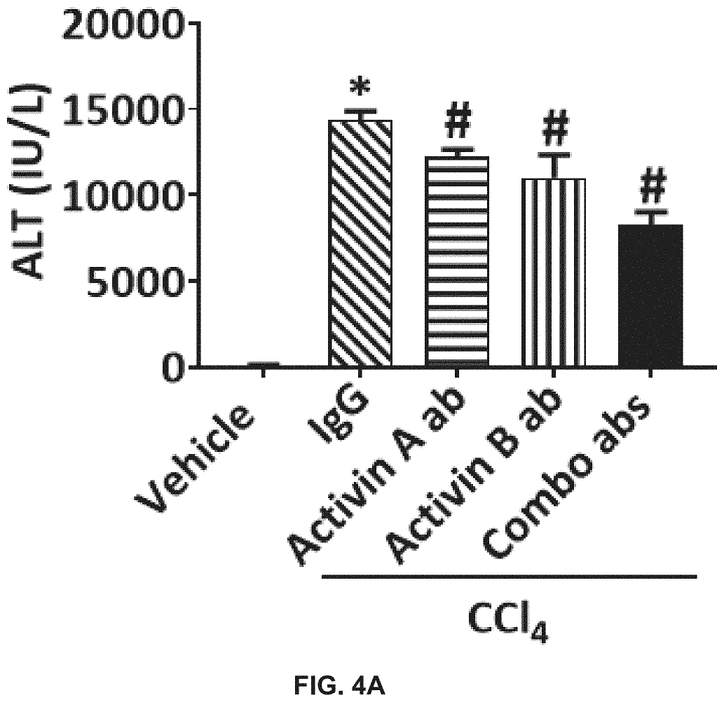

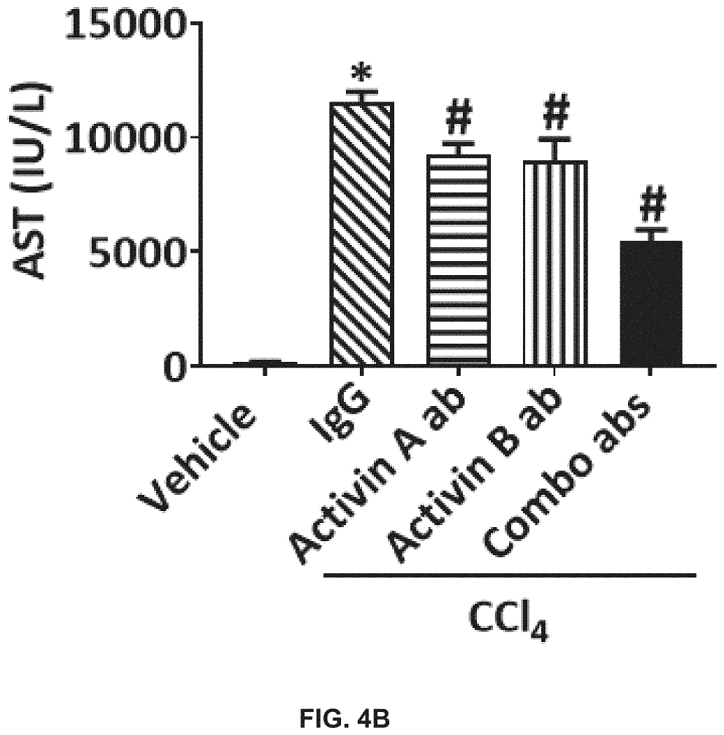

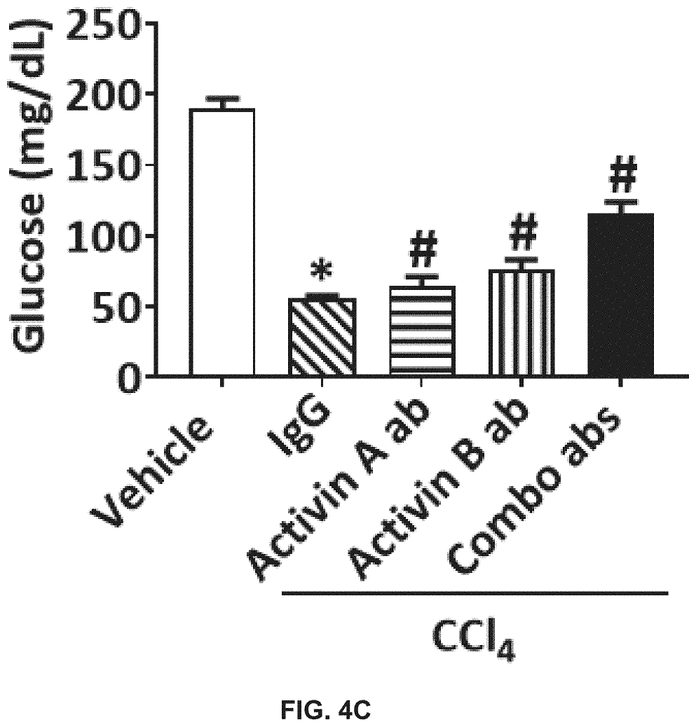

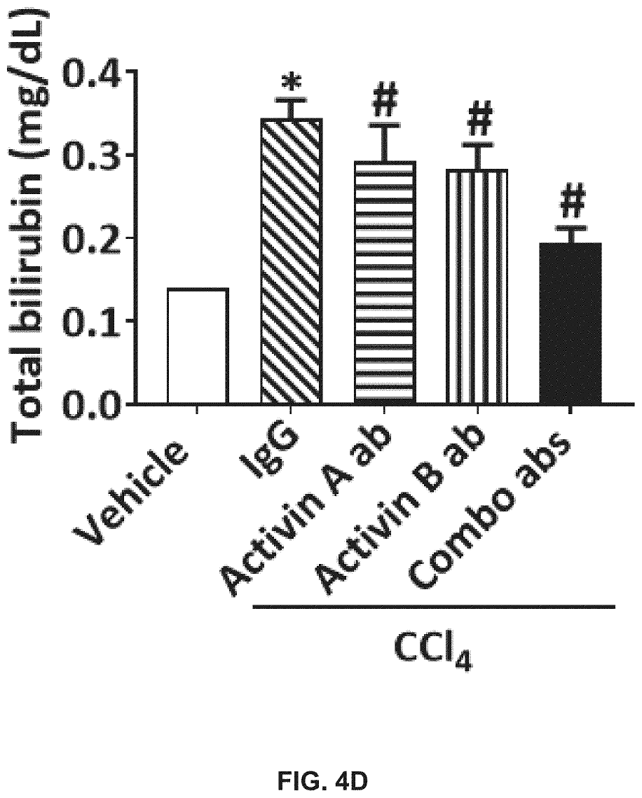

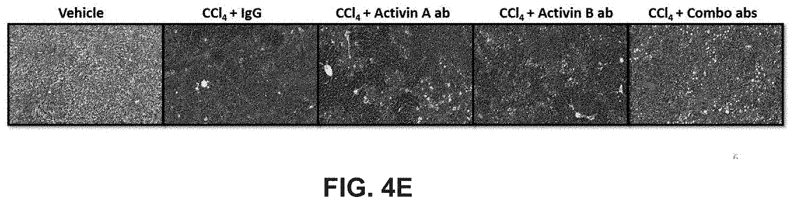

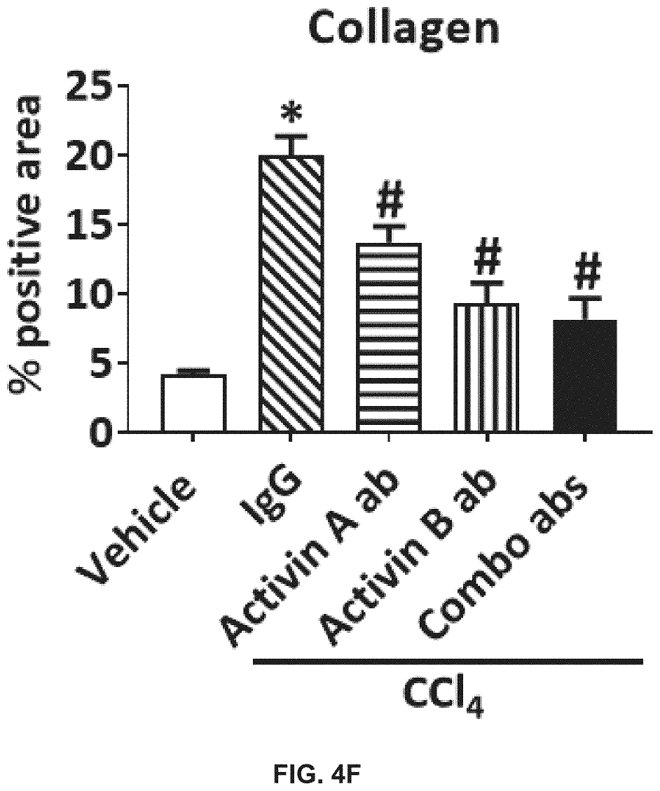

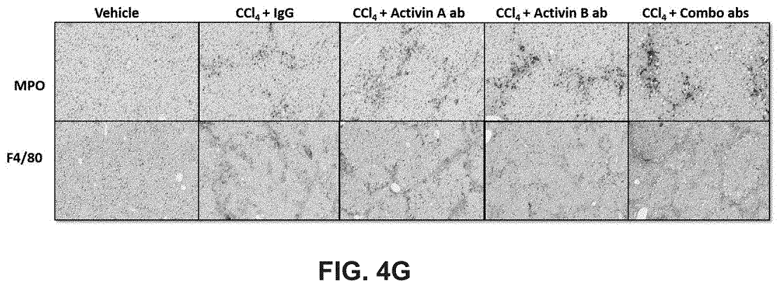

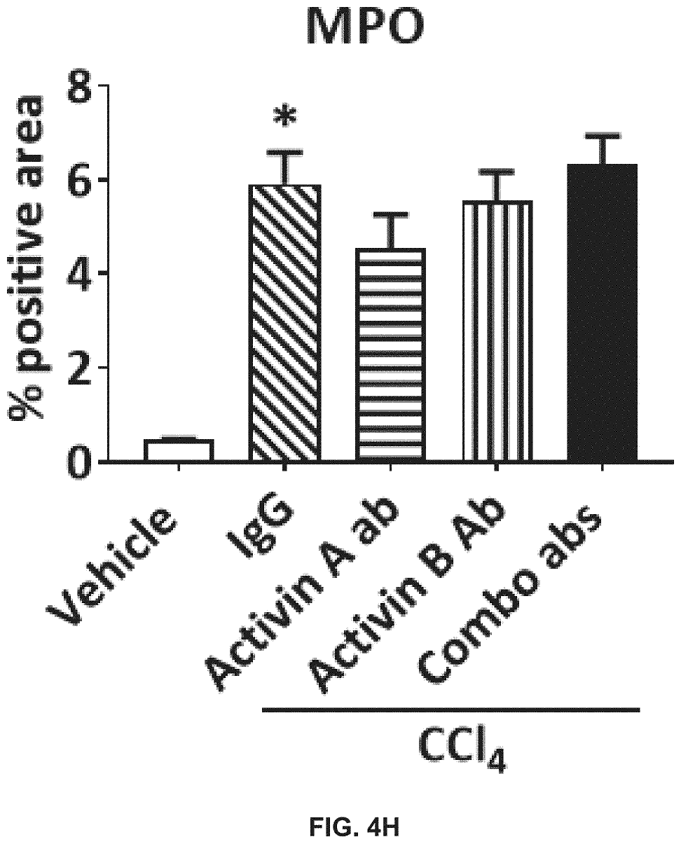

FIGS. 4A-I illustrate that activin B antibody, activin A antibody, and combination of them display different effects in regressing liver fibrosis induced by CCl₄ in mice. Adult female mice were subjected to CCl₄ or vehicle injection (i.p.) twice per week for 10 weeks. Starting from the seventh week, these mice were treated (s.c.) with IgG (60 mg/kg), activin A antibody (10 mg/kg of activin A antibody+50 mg/kg of IgG), activin B antibody (50 mg/kg of activin B antibody+10 mg/kg of IgG), or combination of activin A and activin B antibodies (10 mg/kg of activin A antibody+50 mg/kg of activin B antibody) weekly. As shown in FIG. 4A, ten weeks after the initial CCl₄ injection, ALT in the blood was analyzed. As shown in FIG. 4B, ten weeks after the initial CCl₄ injection, AST in the blood was analyzed. As shown in FIG. 4C, ten weeks after the initial CCl₄ injection, glucose in the blood was analyzed. As shown in FIG. 4D, ten weeks after the initial CCl₄ injection, total bilirubin in the blood was analyzed. Representative liver sections stained with Masson's trichrome are shown in FIG. 4E. Percent Masson's trichrome staining areas were quantified by ImagJ as shown in FIG. 4F. Representative liver MPO and F4/80 immune-histological staining images are shown in FIG. 4G. FIG. 4H shows quantification of percent positive staining area of MPO. FIG. 4I shows quantification of percent positive staining area of F4/80. Data are presented as means±S.E.M. (n=8). *, P<0.05 compared to vehicle controls. #, P<0.05, compared to IgG controls.

Consequently, distinct reversal effects were observed among the antibody treatment groups. The reversal effects followed the sequence of magnitude of effect where inactivating both activin B and A had greater effect than inactivating activin B alone, and inactivating activin A had the lowest effect. Combinational inactivation exerted the most beneficial effects across all assessments, including reduced liver injury (as measured by serum ALT and AST), improved serum glucose and total bilirubin levels, decreased collagen deposition, and less macrophage infiltration (FIGS. 4A-I). Inactivating activin B alone generated a stronger anti-fibrotic effect, but nearly equal effects in other assessments, compared with inactivating activin A alone (FIGS. 4A-I). Of note, inactivating activin B alone and inactivating both activin B and A equivalently regressed liver fibrosis (FIGS. 4E-F). Neutrophils and Kupffer cells were similarly distributed in fibrotic livers, concentrating in septa. Neutralizing activin A, activin B, or both did not alter the total number of infiltrated neutrophils, but almost equally reduced the total number of Kupffer cells (FIGS. 4H-I). This suggests that activin B and activin A essentially cooperate to modulate the functional state of Kupffer cells. Collectively, these results demonstrate that activin B is a stronger driver of liver fibrogenesis than activin A, and that these two activin ligands may act cooperatively during the progression of chronic liver injury. Additionally, neutralization of activin B or both of these ligands largely reverses liver fibrosis, as well as prevent the onset of this pathogenesis.

### Example 5

Activin B is Associated with Hepatocyte Injury and May Induce Hepatocyte Differentiation.

In situ hybridization was performed on formalin-fixed and paraffin-embedded liver sections using mouse inhibin A and inhibin B RNAscope probes and a 2.5 HD Assay-Brown kit according to the manufacturer's protocol (Advanced Cell Diagnostics, Hayward, Calif.). See Bingham et al., “RNAscope in situ hybridization confirms mRNA integrity in formalin-fixed, paraffin-embedded cancer tissue samples,” Oncotarget 2017; 8(55): pp. 93392-403. Briefly, mouse liver sections were subjected to in situ hybridization for inhibin inhibin βB, and TGFβ1. Sections were pretreated using an extended protease treatment and hybridized under conditions as described by the manufacturer by using automated RNAScope probes for activin A, activin B, and TGFβ1, as well as standard negative dihydrodipicolinate reductase (DapB; a bacterial gene) and positive peptidylprolyl isomerase B (PPIB) control probes. The probes were detected using RNAScope LS 2.5 Duplex brown Assay for the Leica Bond RX autostainer (Cat. no. 322440) and Brown DAB (Cat. no.DS9800). Slides were counter-stained with hematotoxin.

The cellular sources of activin B were revealed by in situ hybridization. Activin B and A were mainly transcribed in hepatocytes and biliary epithelial cells in vehicle-controlled livers and additionally in fibrogenic cells in the fibrotic livers (FIG. 2I). Activin B is associated with hepatocyte injury and may induce hepatocyte differentiation. In situ hybridization showed that hepatocytes are the main cellular source in the liver that expresses inhibin βB and inhibin mRNAs (FIG. 2I).

### Example 6

Secretion of Activin B and a Proteins by Hepatocytes and Response to Hepatocyte Injury

To examine whether activin B and A proteins are secreted by hepatocytes and how these proteins respond to hepatocyte injury, primary mouse hepatocytes (PMH) were exposed to CCl₄ or lipopolysaccharide (LPS). Primary mouse hepatocytes (PMHs) were isolated and grown from adult male C57Bl/6 mice, as described previously. See Akie et al., “Determination of Fatty Acid Oxidation and Lipogenesis in Mouse Primary Hepatocytes,” J Vis Exp 2015(102): p. 5298. Briefly, under anesthesia, the peritoneal cavity was opened, and the liver was perfused in situ via the portal vein for 4 min at 37° C. with calcium-magnesium (CM)-free HEPES buffer and for 7 min with CM-free HEPES buffer containing Type IV collagenase (35 mg/100 mL) and CaCl2 (10 mM). Cells were used only if the cell viability was above 90% as assessed by trypan blue exclusion. After three centrifugations (44 g for 2 min) in Leibovitz's L-15 washing media supplemented with 0.2% bovine albumin, cells were plated onto 24-well or 96-well plates (26,000 cells/cm2). Cells were cultured in high-glucose (25 mM) DMEM supplemented with 10% FBS. All culture media contained penicillin (100 units/ml) and streptomycin (100 μg/ml). After cell attachment for 2 h, the medium was replaced with fresh medium supplemented with 10% fetal bovine serum (FBS). PMH cultures were maintained under 5% CO2 atmosphere at 37° C. RAW264.7 cells, a mouse macrophage cell line, were purchased from American Type Culture Collection (Manassas, Va.) and cultured following manufacturer's manual. LX-2 cells, a human hepatic stellate cell line, were cultured in DMEM supplemented with 2% FBS (Gibco, Invitrogen, Carlsbad, Calif.). These cells were treated with activin A, activin B, activin C, CXCL1, and TGFβ1.

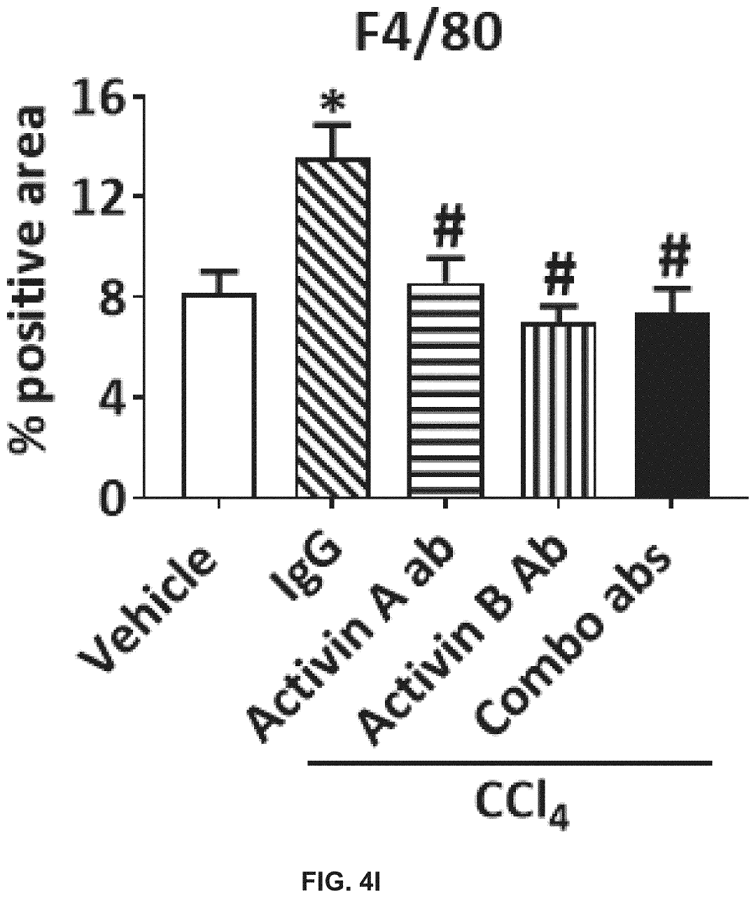

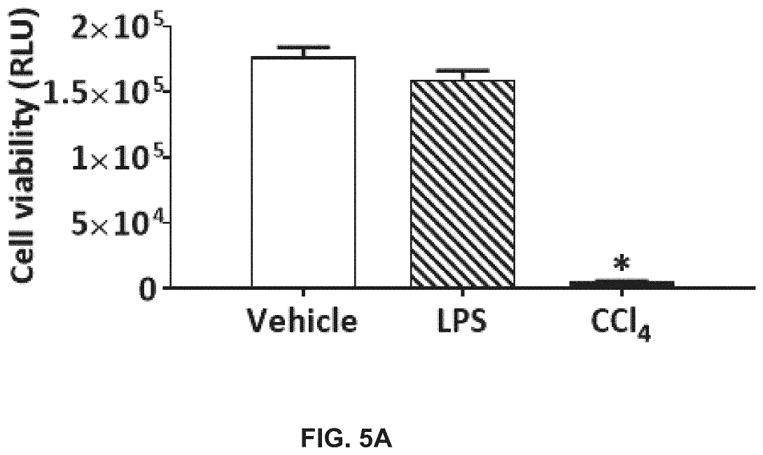

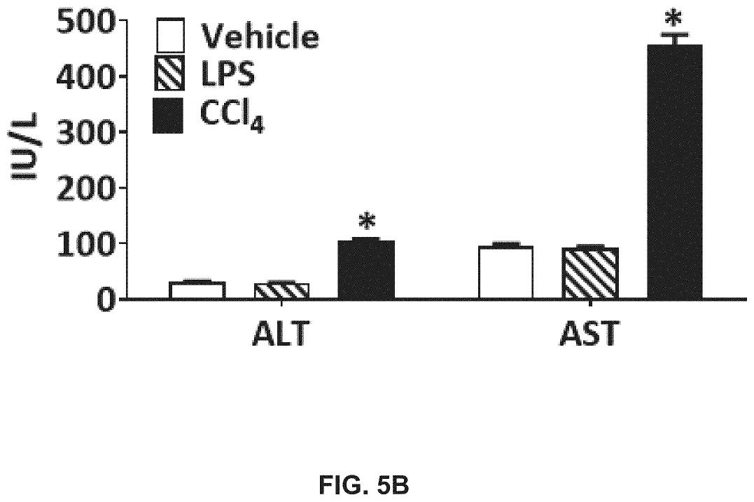

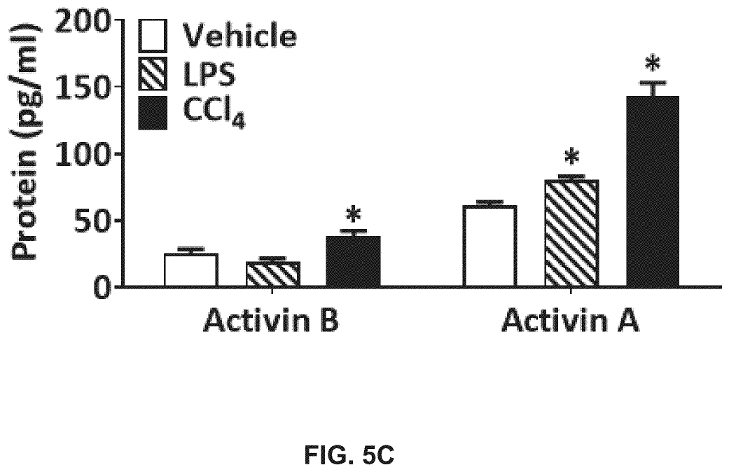

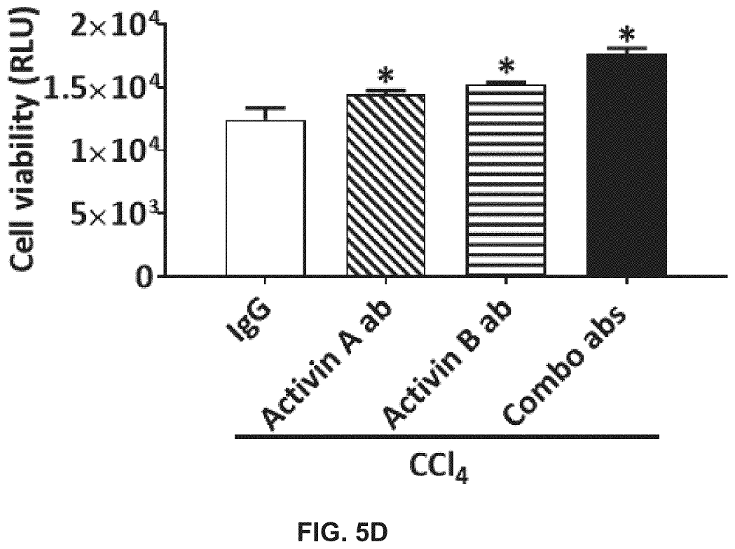

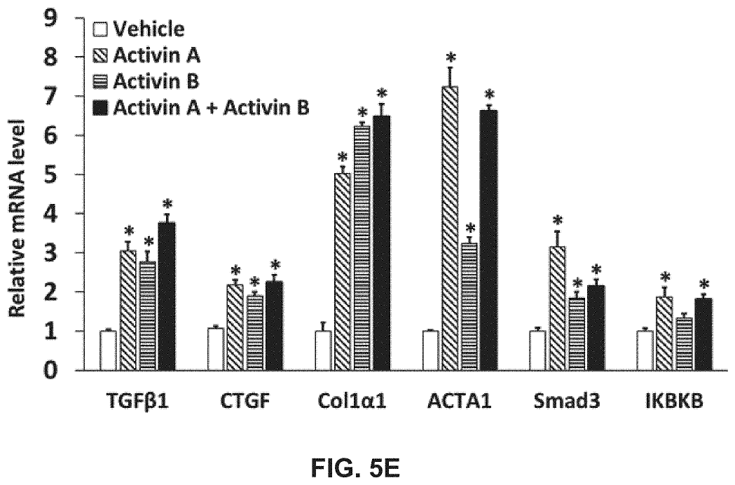

FIGS. 5A-E illustrate that activin B is produced in primary mouse hepatocytes (PMHs) and induces differentiation of these cells. PMHs were isolated from adult male mice and cultured overnight. Subsequently, the cells were treated with vehicle (corn oil), lipopolysaccharide (LPS, 10 μg/ml), or 0.5% CCl₄ for 24 hours. As shown in FIG. 5A, cell viability was analyzed. As shown in FIG. 5B, supernatant ALT and AST were analyzed. As shown in FIG. 5C, supernatant activin A and B proteins were analyzed. FIG. 5D shows cell viability of primary hepatocytes after treatment for 24 hours with 0.5% CCl₄ and co-treatment with IgG, activin A antibody, activin B antibody, or combination of both antibodies (100 ng/ml each). FIG. 5E shows the mRNA levels of the genes indicated were evaluated by qRT-PCR in PMHs treated with activin A and B (100 ng/ml each), or their combination for 24 hours. For all above assays, data are expressed as means S.E.M. *, P<0.05 vs. vehicle controls.

CCl₄ damaged these cells and induced their necrosis, which was indicated by increased release of ALT and AST into the culture supernatants (FIGS. 5A-B), as well as increased the secretion of both activin B and A proteins (FIG. 5C). Cell viability was marginally, significantly, and additively improved by neutralizing activin A, activin B, and the combination of both, respectively (FIG. 5D). In contrast, LPS only stimulated PMH to increase activin A production without affecting activin B, ALT and AST (FIG. 5C). Hence, hepatocytes are one of the cellular sources responsible for secretion of activin B and activin A and exhibit toxin-dependent responses. Moreover, activin B production in hepatocytes is accompanied with hepatocyte injury and cell death. Of note, these two activins modulate hepatocyte injury, as neutralization of these proteins improved cell viability following insults. PMH responded to individual or combinational treatment of these two proteins by equivalently upregulating the transcription of TGFβ1, CTGF, and Col1α1, as well as distinctly regulating the mRNA expression of ACTA1, Smad3, and IKBKB (FIG. 5E). These genes were associated with myofibrolast activity, which suggest that activin B and A may be involved in hepatocyte differentiation into myofibrolast-like cells following injury. Thus, activin B and A have redundant, specific, and interactive actions in hepatocytes.

### Example 7

Activin B Directly Targets Macrophages and Modulates Inflammatory Cytokines.

Immune cells centrally mediate inflammation largely via regulating cytokine production. In liver, macrophages are contributed by local Kupffer cells and infiltrated monocytes from circulation. RAW264. 7 macrophages are widely used in hepatic inflammation studies. See Chen et al., “Pirfenidone prevents and reverses hepatic insulin resistance and steatohepatitis by polarizing M2 macrophages,” Nature Lab Invest 2019; 99: pp. 1335-1348. Briefly, RAW264.7 cells, a mouse macrophage cell line, were cultured following their manufacturer's manual. They were then exposed to activin B or/and activin A and evaluated for the expression of inflammatory cytokines or chemokines.

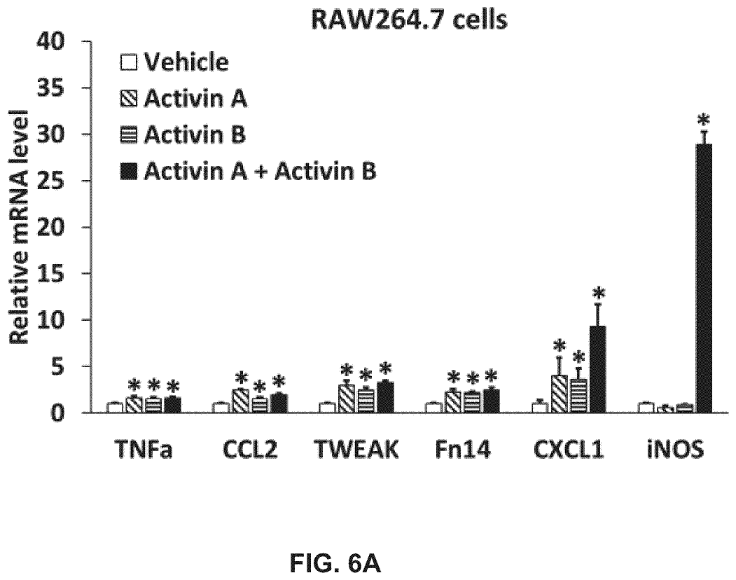

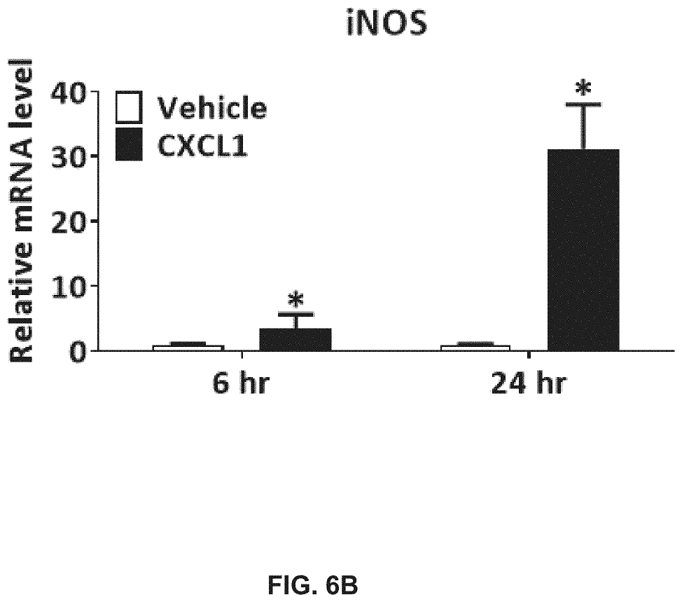

FIGS. 6A-B illustrate that activin B induces macrophages to express inflammatory cytokines or chemokines. FIG. 6A shows transcripts of the several genes indicated were quantified by qRT-PCR in RAW264.7 cells after exposure to activin A (100 ng/ml), activin B (100 ng/ml), or their combination (100 ng/ml each) for 24 hours. FIG. 6B shows the mRNA expression of iNOS was evaluated with qRT-PCR in RAW264.7 cells following vehicle or CXCL1 (100 ng/ml) treatment for 6 or 24 hours. For above quantitative analyses, data are presented as means±S.E.M. *, P<0.05 vs. vehicle controls.

Individual or combination of both ligands exerted similar potency in upregulating TNFα, CCL2, TWEAK, and Fn14 expression (FIG. 6A), indicating that activin B and A have redundant actions on macrophages. Notably, individual ligand treatments equally, whereas combination ligand treatment additively, elevated chemokine (C-X-C motif) ligand 1 (CXCL1) transcript level (FIG. 6A). Moreover, inducible nitric oxide synthase (iNOS) activation was only seen with exposure to both ligands (FIG. 6A). The additive increase in CXCL1 expression after exposure to both ligands was necessary to achieve iNOS activation, whereas either ligand alone was not able to do so. RAW264.7 cells were treated with CXCL1 protein. Consequently, CXCL1 upregulated iNOS expression by 30-fold after 24 hours treatment (FIG. 6B). Activin B and A directly target macrophages and additively stimulate sufficient production of autocrine CXCL1 to induce iNOS transcription. The existence of an activin B/activin A/CXCL1/iNOS signaling pathway modulating macrophage activity, further supporting the notion that activin B and A essentially collaborate with each other to activate the gene transcription of a subset of inflammatory cytokines and chemokines.

### Example 8

Activin B Directly Promotes Hepatic Stellate Cell (HSC) Activation.

Myofibroblasts centrally drive liver fibrogenesis and are primarily differentiated from activated HSCs. The human HSC cell line LX-2 has been widely used to study the function of HSCs. Because LX-2 cells are more relevant to humans than mouse HSCs, the behavioral responses of LX-2 cells to activin A, activin B, combination of both, and TGFβ1, a recognized regulator of HSC activity were tested.

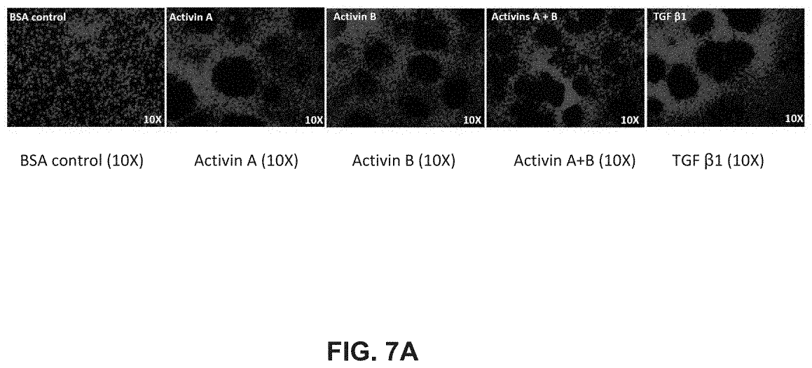

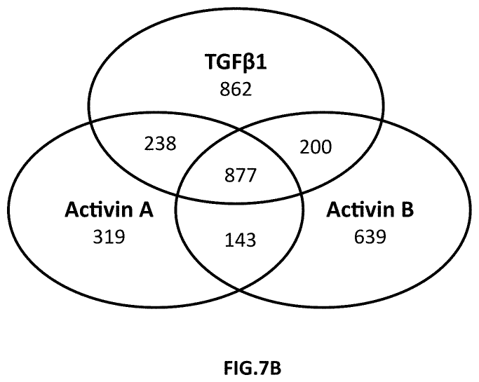

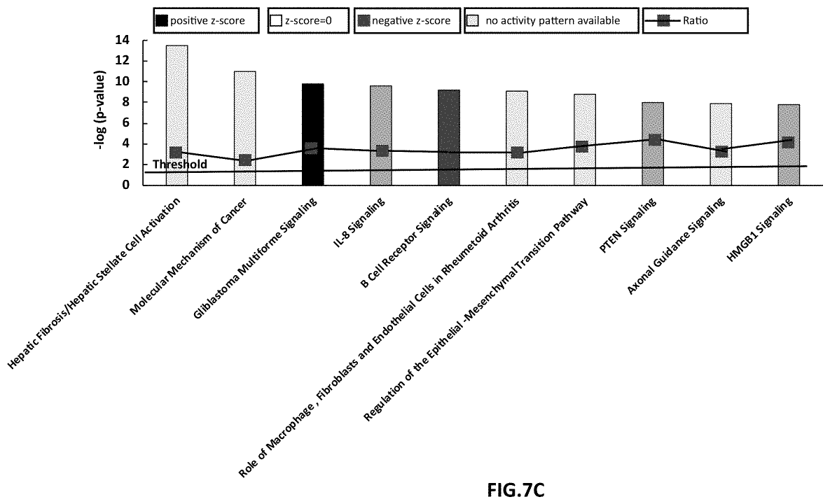

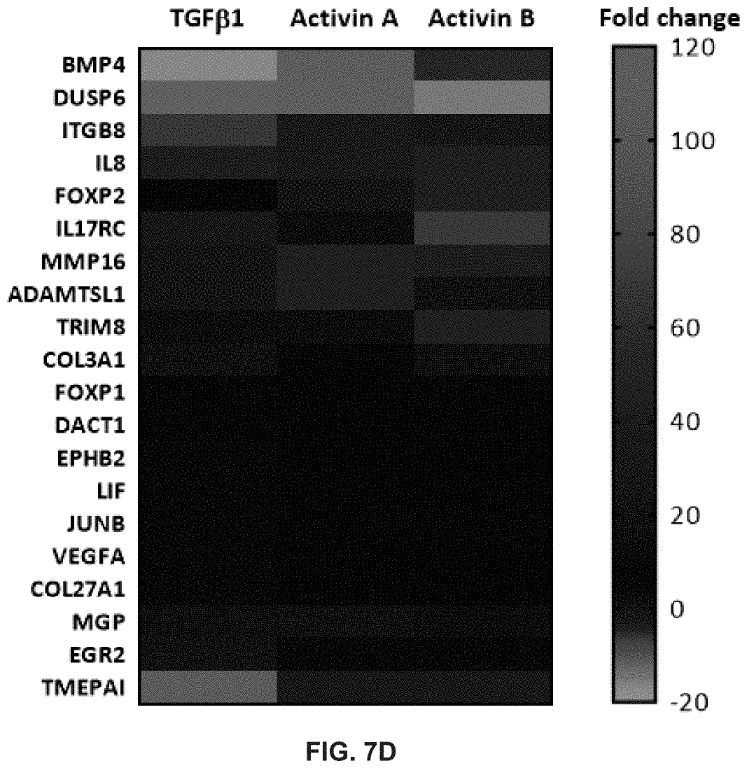

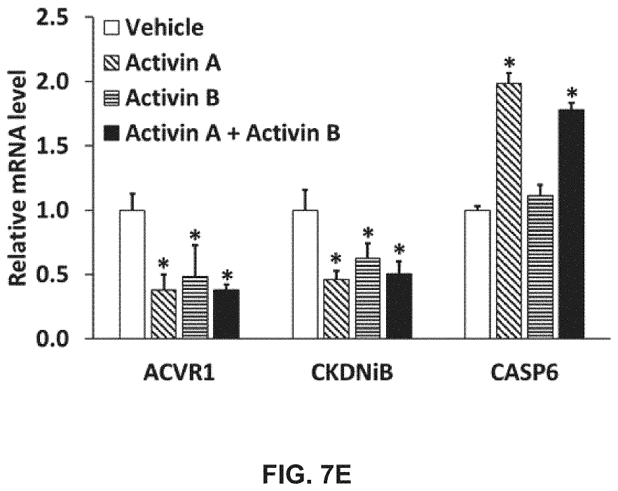

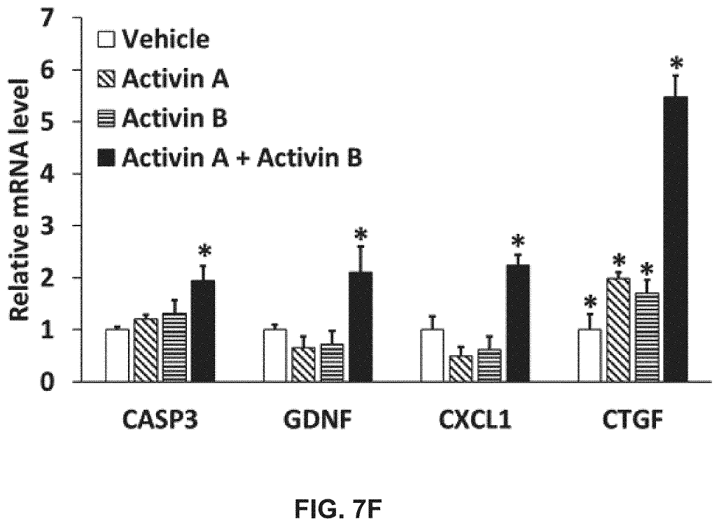

FIGS. 7A-F illustrate that activin B morphologically and molecularly activates HSCs. As shown in FIG. 7A, LX-2 cells were treated with bovine serum albumin (BSA, 100 ng/ml), activin A (100 ng/ml), activin B (100 ng/ml), their combination (100 ng/ml each), or TGFβ1 (5 ng/ml) for 24 hours and then underwent 4′,6-diamidino-2-phenylindole (DAPI) staining. As shown in FIG. 7B, LX-2 cells were treated with activin A (100 ng/ml), activin B (100 ng/ml), or TGFβ1 (5 ng/ml) for 6 hours. Total RNAs were isolated, reverse transcribed to cDNA, and then subjected to microarray analysis using HG-U133 plus 2 chips (n=6). Pie chart shows the numbers of genes commonly or uniquely regulated by the individual ligands. FIG. 7C shows the top ten signaling pathways revealed by Ingenuity Canonical Pathway analysis of the 877 target genes shared by these three ligands. FIG. 7D shows a heat-map of the 20 genes exhibiting the highest magnitudes of upregulation or downregulation in response to these three ligands. FIG. 7E and FIG. 7F show LX-2 cells were treated with vehicle, activin A (100 ng/ml), activin B (100 ng/ml), or their combination (100 ng/ml of each) for 24 hours. The expression of the genes indicated was assessed with qRT-PCR. Data are shown as means of fold changes relative to vehicle controls±S.E.M. *, P<0.05.

LX-2 cells formed a septa-like structure following 24 hours exposure to these three ligands (FIG. 7A), a common behavior observed in HSCs during liver fibrogenesis. Activin B and A directly activate HSCs. LX-2 cells were treated with activin A, activin B, or TGFβ1 protein for six hours, and profiled their early responsive genes by microarray analysis. These three proteins regulate overlapping but differential gene networks (FIG. 7B). The overlapping 877 genes were associated predominately with HSC activation and hepatic fibrosis, including upregulated TGFβ signaling negative feedback modulator transmembrane prostate androgen-induced protein (TMEPAI), early growth response protein 2 (EGR2), calcium ion-binding protein matrix gla protein (MGP), and downregulated BMP4, dual specificity phosphatase 6 (DUSP6), extracellular matrix glycoprotein TNXB, IL-8, and IL-17 receptor C (FIGS. 7C-D). Activin signaling dictates a spectrum of HSC properties redundantly via multiple ligands including activin A and B. On the other hand, each of these individual ligands has a large and unique set of target genes associated with critical cellular functions. For instance, activin B exclusively decreased cell migration-associated scaffold protein Ezrin and calcium-dependent phospholipid-binding protein 3, implying a role for activin B in controlling HSC migration. Thus, activin B is a novel direct regulator of HSCs and that activin ligands distinctly but coordinately regulate the transcriptome of HSCs.

To gain insight into how activin A and B interactively act on HSCs, LX-2 cells were exposed to activin A or B alone or both and subsequently examined how a group of genes known to regulate HSC activity respond transcriptionally. Four scenarios were observed: (1) ACVR1 and CKDNiB equivalently responded to individual ligand (FIG. 7E); (2) CASP6 solely responded to activin A (FIG. 5E); (3) CASP3, GNDF, and CXCL1 specifically responded to dual ligands (FIG. 7F); and (4) CTGF equally responded to individual ligands but synergistically to dual ligands (FIG. 7F). The results indicate that activin B and A have redundant, unique, and interactive effects on HSCs. Taken together, these in vitro data demonstrate that activin B and A elicit both redundant and interactive modulation of HSCs.

Discussion of Example 8

Circulating activin B is closely associated with liver injury and fibrosis regardless of etiologies and species, suggesting a highly conserved, activin B-mediated mechanism of response to liver insult in mammals. This response is activated rapidly following liver injury, operates stably throughout progression of disease, and predominates until the injured liver becomes fibrotic. Inhibin βB mRNA was abundantly transcribed in fibrotic cells in chronically injured livers, and elevated hepatic activin B protein was always concomitant with enriched circulating activin B protein. Therefore, it is likely that increased production of hepatic activin B largely contributes to its systemic elevation during liver fibrosis development.

Activin B acts as a potent driver of the collective complications (hepatocyte injury, inflammation, and fibrosis) of chronic liver injury. Activin B mediates hepatocyte injury and may induce hepatocyte differentiation into myofibrolast-like cells. Activin B regulates the activities of macrophages. For example, activin B upregulated TWEAK and its receptor Fn14 in macrophages. The TWEAK/Fn14 pathway has been shown to promote reactive oxygen species (ROS) production and oxidative stress in macrophages, leading to hepatocyte apoptosis and HSC activation. TWEAK is known to be primarily produced by macrophages and induces the expansion of liver progenitor cells; it mediates cross-talk among liver progenitor cells, immune cells, and HSCs, thereby augmenting inflammatory and fibrotic responses in chronically injured liver. Some of the modulatory effects of activin B during liver injury and fibrogenesis may be mediated through an activin B/TWEAK/Fn14 axis operating in multiple liver cell populations. Activin B acts as a potent pro-fibrotic factor during liver injury. In vitro, activin B massively altered the transcriptome of HSCs towards a pro-fibrotic myofibrolast-like profile and induced septa-like structure formation. Behavioral features of these cells were revealed, offering an in vitro assay to investigate the regulation of HSC migration and morphology in fibrotic liver. Microarray data have provided a list of activin B target genes of interest to elucidate how activin B modulates HSC activity during liver injury. In vivo, neutralizing activin B alone largely repressed septa formation, collagen deposition, and expression of fibrotic genes such as CTGF and TGFβ1 in chronically injured liver. Activin B promotes the initiation and progression of liver fibrosis by facilitating hepatocyte injury, augmenting inflammation, and sustaining HSC activation.

Increased activin B requires the presence of activin A to optimally mediate liver injury progression. Hepatic and circulating activin A were only transiently increased during the acute phase of liver injury and were kept at pre-injury levels throughout the long-term chronic phase. However, neutralizing activin A alone did produce beneficial effects, although less than neutralizing activin B alone, in both the prevention and reversal studies. Furthermore, upregulation of CTGF and TGFβ1 requires both activin B and A in chronically damaged livers. Most strikingly, inactivating both activin B and activin A gave rise to the most profound beneficial effects across hepatic structural and functional assessments compared to inactivating activin B or A alone. As liver injury progresses, elevated activin B needs constitutive activin A for cooperative pathological effects, as both ligands are required for activating certain cellular programs that otherwise would not be initiated by a single ligand. This represents a novel mode of action of activin ligands in general and a new mechanism governing the actions of activin B and A specifically. Additionally, a number of additive or interdependent effects between activin B and activin A in vitro in macrophages and HSCs as well as in vivo in chronically fibrotic livers were observed. For example, both activin B and A are required for sufficient upregulation of CXCL1 to activate iNOS in macrophages. This provides important mechanistic insight into the actions of activin B and A, as iNOS is highly inducible in response to immunological stimuli and is known to promote many pathological processes including liver fibrosis.

Increasing understanding of TGFβ signaling in the pathophysiology of fibrosis has prompted preclinical and clinical efforts targeting its ligands, receptors, or Smads ultimately for therapeutic benefits. However, few of these efforts have resulted in positive patient outcomes largely due to off-target complications. So far, Pirfenidone is the only small molecule TGFβ signaling inhibitor approved for treating human idiopathic pulmonary fibrosis and has significant side effects in the GI tract and the skin. Neutralization of activin B alone or both of activin B and A did show exciting therapeutic efficacy. Pre-clinical studies demonstrate that targeting activin B or ideally both activin B and A is a promising strategy to prevent and even reverse liver fibrosis.

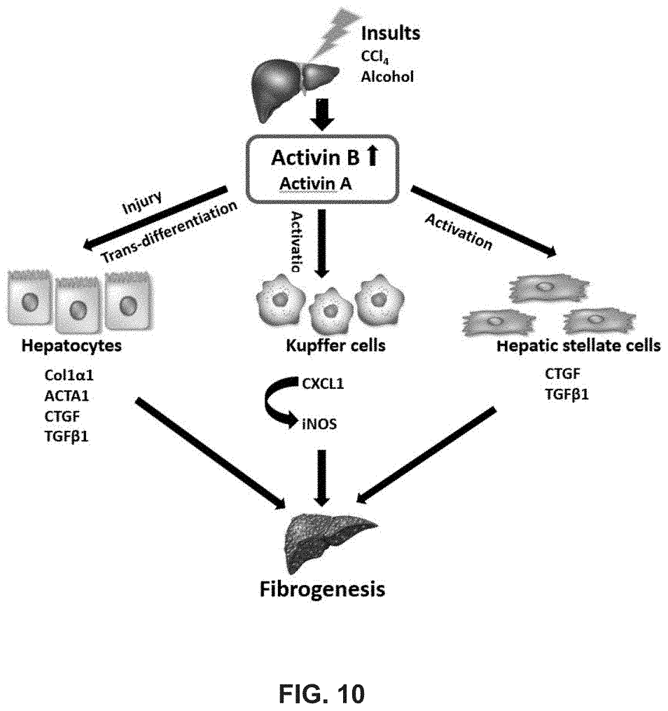

In summary, activin B was identified as a potent driver and a promising therapeutic target of liver fibrosis. Though not wishing to be bound by any theory, it is proposed that, with reference to FIG. 10 which illustrates a scheme of how Activin B and A regulate the initiation and progression of liver fibrosis, regardless of etiology, injured hepatocytes constantly produce activin B; increased activin B cooperates with constitutive activin A to induce hepatocyte injury and possible differentiation in an autocrine and paracrine manners, to modulates macrophages to secrete inflammatory cytokines through CXCL1/iNOS, TWEAK/Fn14, and other signaling pathways, and, most critically, to initiate and maintain the activation of HSCs by increasing the expression of profibrotic genes including CTGF and TGFβ1. This hypothesis highlights activin B as a potent promoter of liver fibrogenesis and lays the groundwork for future investigations to elucidate how activating B acts in various liver cell populations during the initiation and progression of liver fibrosis.

### Example 9

Activin B, Activin a and their Combination Promotes the Initiation and Progression of Chronic Liver Injury

To further validate the findings using the CCl₄ model, a similar study in BDL-induced chronic liver injury was conducted in mice. Experimental groups included (1) sham control; (2) IgG+BDL; (3) Activin A antibody+BDL; (4) Activin B antibody+BDL; and (5) Activin A antibody+Activin B antibody+BDL. The first antibody dosing was done one day prior to BDL surgery and the second dosing one week after BDL. Endpoint analyses were conducted two weeks post-BDL.

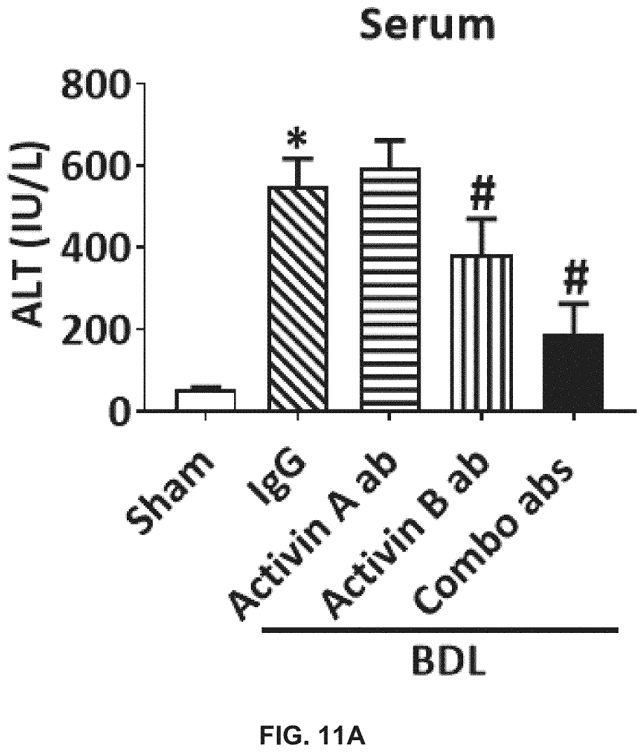

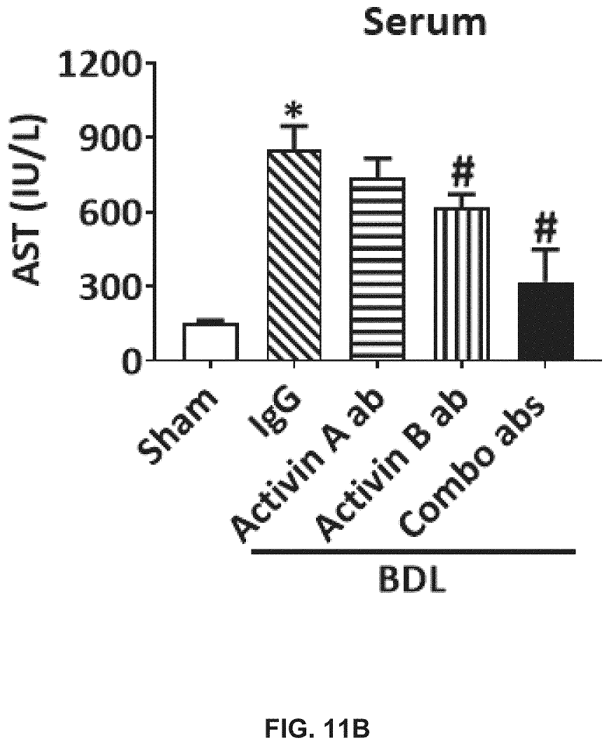

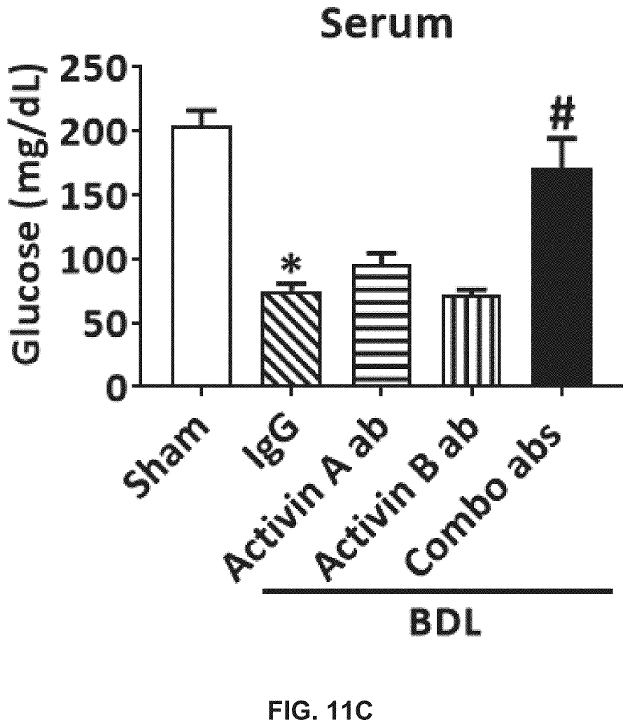

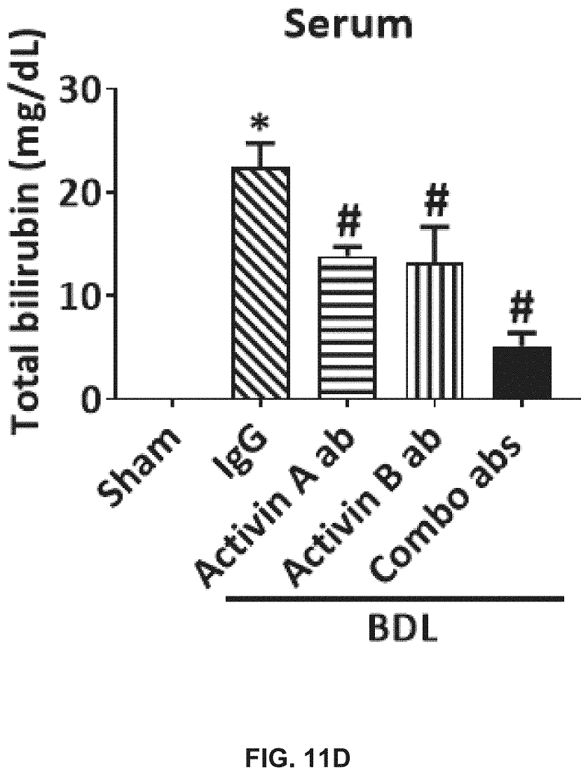

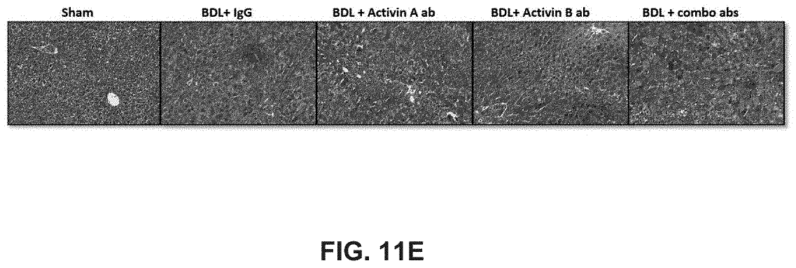

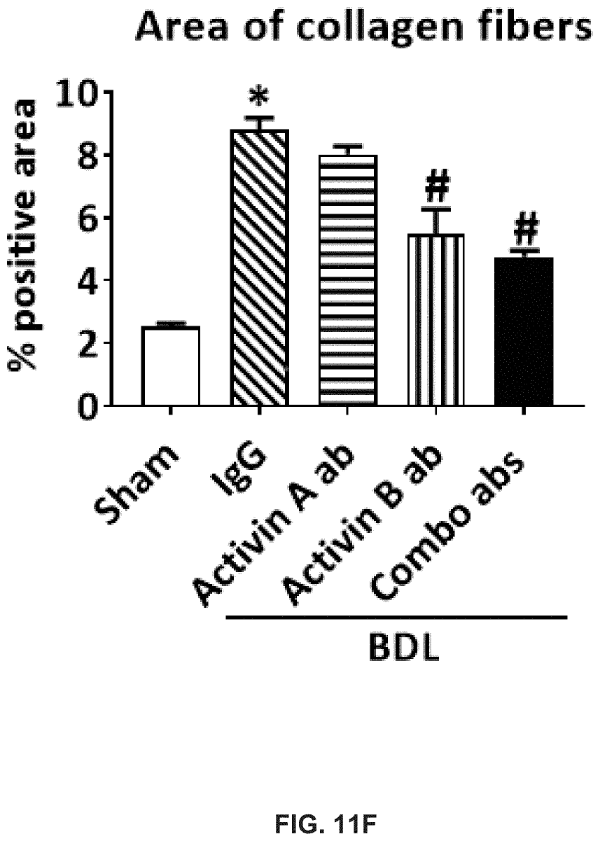

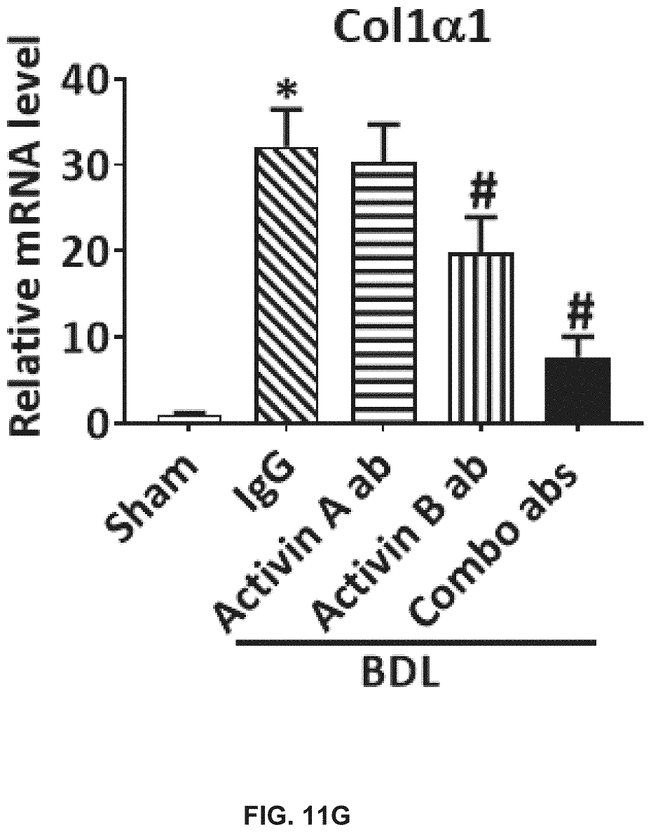

FIGS. 11A-G illustrate that activin B antibody, activin A antibody, and combination of them show distinct effects in preventing liver fibrosis induced by BDL in mice. Experimental groups included (1) sham control; (2) IgG+BDL; (3) Activin A antibody+BDL; (4) Activin B antibody+BDL; and (5) Activin A antibody+Activin B antibody+BDL. The first antibody dosing was performed one day prior to BDL surgery and the second dosing one week after BDL. As shown in FIG. 11A, two weeks after BDL, ALT in the blood was analyzed. As shown in FIG. 11B, two weeks after BDL, AST in the blood was analyzed. As shown in FIG. 11C, two weeks after BDL, glucose in the blood was analyzed. As shown in FIG. 11D, two weeks after the BDL, total bilirubin in the blood was analyzed. Two weeks after BDL, representative liver sections stained with Masson's trichrome are shown in FIG. 11E. Two weeks after BDL, percent Masson's trichrome staining areas were quantified by ImagJ are shown in FIG. 11F. FIG. 11G shows the mRNA expression of hepatic Col1α1 was evaluated by qRT-PCR.

It was found that activin B antibody treatment reduced liver injury (FIGS. 11A-11B), improved liver function (FIGS. 11C-11D), and decreased liver fibrosis (FIGS. 11E-11G). However, activin A antibody did not show beneficial effects in nearly all the endpoints analyzed except that it ameliorated total bilirubin index (FIGS. 11A-11G). Remarkably, the dual antibodies exerted the most prominent efficacy, manifested by reduced liver injury, improved liver functions, and decreased liver fibrosis (FIGS. 11A-11G).

These hypotheses were tested further in bile duct-litigated mice using procedures described previously. See Tag et al., “Bile duct ligation in mice: induction of inflammatory liver injury and fibrosis by obstructive cholestasis,” J Vis Exp 2015; (96): p. 52438. Bile duct litigated induced liver fibrosis was characterized by proliferation of cholangiocytes, upregulation of cholangiocyte-specific marker cytokeratin 19 (CK19), and increased expression of fibrogenic markers such as α-SMA, collagen I, and TGFβ1.

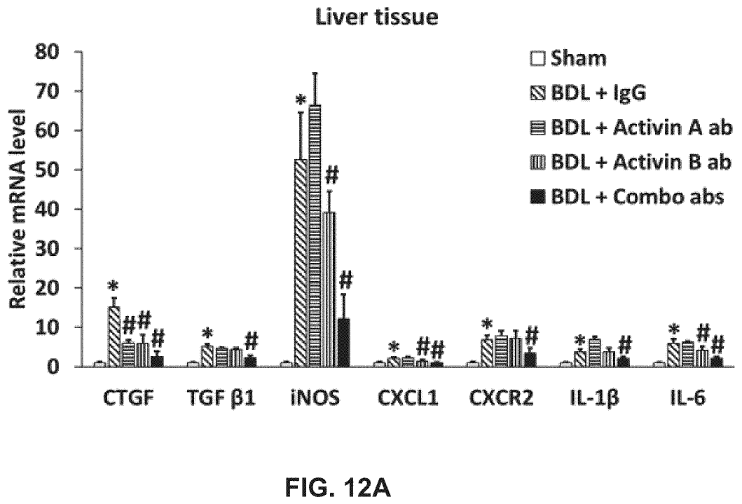

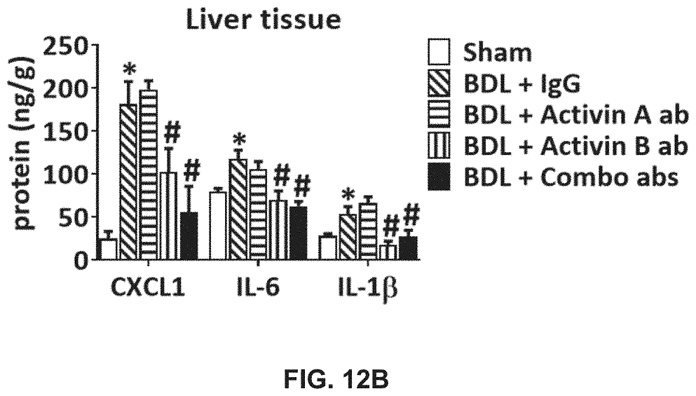

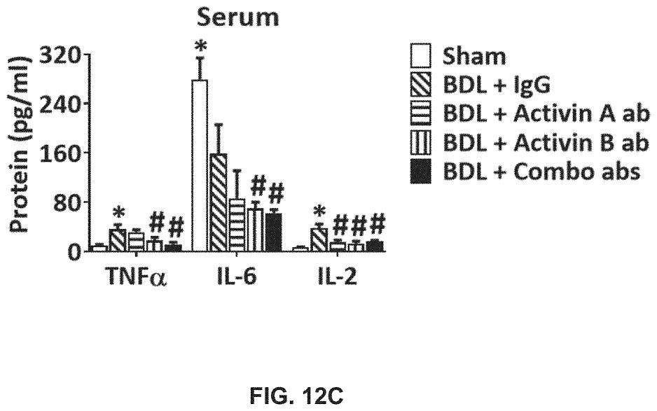

FIGS. 12A-C illustrate that Activin B strongly, but Activin A weekly, modulate local and systemic inflammatory cytokines and pro-fibrotic factors during chronic liver injury. FIG. 12A shows that transcripts of the genes indicated were quantified by qRT-PCR in liver tissues. Activin B or dual antibody treatment inhibited the mRNA expression of hepatic CTGF, TGFβ1, iNOS, CXCL1, CXCR2, IL-01, and IL-6, whereas Activin A antibody treatment only suppressed CTGF Furthermore, in bile duct-ligated mice, Activin B antibody or dual antibody treatment reduced CXCL1, IL-6, and IL-10 protein contents in the livers as well as IL-6, TNFα, and IL-2 protein concentrations in the circulation, to similar or distinct extents, whereas activin A antibody treatment merely decreased the amount of circulating IL-2 protein in the livers (FIG. 12B) and the serum (FIG. 12C).

Thus, using bile duct-ligated mice, activin B antibody or dual antibody treatment reduced CXCL1, IL-6, and IL-10 protein contents in the livers as well as IL-6, TNFα, and IL-2 protein concentrations in the circulation, to similar or distinct extents, whereas activin A antibody treatment merely decreased the amount of circulating IL-2 protein (FIGS. 12B-12C). These molecules are known promoters of liver inflammation and fibrosis. These data demonstrate that activin B strongly, but activin A weekly, modulate local and systemic inflammatory cytokines and pro-fibrotic factors during chronic liver injury.

Collectively, the results demonstrate that, in this model, (1) activin B strongly, but activin A minimally, promotes the initiation and progression of chronic liver injury; (2) the presence of activin A enhances the promoting actions of activin B, underlying why the dual targeting approach is the most beneficial; and (3) inactivating activin B or both activin B and A is an effective approach to prevent and even treat liver fibrosis and chronic liver injury.

As will be appreciated from the descriptions herein, a wide variety of aspects and embodiments are contemplated by the present disclosure, examples of which include, without limitation, the aspects and embodiments listed below:

A method is presented for modulating liver fibrosis in a subject by suppressing hepatic inflammation caused by an hepatic inflammatory response. The methods of the invention comprise administering an agent that inhibits activin, i.e. an activin inhibitor.

A method in accordance with any embodiment disclosed herein, wherein the activin to be inhibited is activin A. In other embodiments, the activin to be inhibited is activin B. In still other embodiments both activin A and activin B are inhibited.

A method in accordance with any embodiment disclosed herein, wherein the activin inhibitor may be an activin A antibody.

A method in accordance with any embodiment disclosed herein, wherein the activin inhibitor may be an activin B antibody.

A method in accordance with any embodiment disclosed herein, wherein antibody may be bivalent, inhibiting both activin A and activin B.

A method in accordance with any embodiment disclosed herein, wherein the activin inhibitor is an antibody fragment.

A method in accordance with any embodiment disclosed herein, wherein the activin inhibitor is a polypeptide.

A method in accordance with any embodiment disclosed herein, wherein the activin inhibitor may be a composition where the composition may include either an antibody or antibody fragment for activin A or an antibody or antibody fragment for activin B.

A method in accordance with any embodiment disclosed herein, wherein the composition comprises both an antibody or antibody fragment for activin A and an antibody or antibody fragment for activin B.

A method in accordance with any embodiment disclosed herein, wherein the activin inhibitor or inhibitors is administered once per day. In other embodiments, the activin inhibitor is administered two or more times daily. In other preferred embodiments, the methods include wherein the activin A or the antibody B antibody may be administered more than once per week.

A method in accordance with any embodiment disclosed herein, wherein the composition containing the activin inhibitor or inhibitors is administered one time daily. In other embodiments, composition is administered two or more times daily. In other preferred embodiments, the composition may be administered one or more times per week.

A method in accordance with any embodiment disclosed herein, wherein the administration of the activin inhibitor or inhibitors or composition containing the same occurs by any pharmaceutically or medically conventional means into the subject.

A method in accordance with any embodiment disclosed herein, wherein the the activin inhibitor or inhibitors or composition containing the same are injected at a single site per dose or multiple sites per dose.

A method in accordance with any embodiment disclosed herein, wherein all of the materials can be packaged into a kit containing all of the necessary components to carry out the claimed methods.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

All publications, patents and patent applications referenced herein are hereby incorporated by reference in their entirety for all purposes as if each such publication, patent or patent application had been individually indicated to be incorporated by reference.

1. A method of modulating liver fibrosis in a subject by suppressing hepatic inflammation caused by a hepatic inflammatory response comprising administering an activin inhibitor.

2. The method of claim 1, wherein the activin to be inhibited is activin A.

3. The method of claim 1, wherein the activin to be inhibited is activin B.

4. The method of claim 1, wherein the activin to be inhibited is both activin A and activin B.

5. The method of claim 1, wherein the activin inhibitor is an activin A antibody or antibody fragment thereof.

6. The method of claim 1, wherein the activin inhibitor is be an activin B antibody or antibody fragment thereof.

7. The method of claim 1, wherein the activin inhibitor is a bivalent antibody or antibody fragment thereof directed against both activin A and activin B.

8. The method of claim 1, wherein the activin inhibitor is an activin polypeptide.

9. A method of preventing or treating liver fibrosis in a subject by suppressing hepatic inflammation comprising administering a pharmaceutical composition comprising at least one activin inhibitor, wherein the activin inhibitor is one or more of an activin A antibody or antibody fragment thereof, an activin B antibody or antibody fragment thereof.

10. The method of claim 9, wherein the pharmaceutical composition further comprises at least one pharmaceutically acceptable excipient.

11. The method of claim 10, wherein the pharmaceutically acceptable excipient comprises one or more of a pharmaceutically acceptable carrier, diluent, stabilizer, surfactant, or buffering agent.

12. The method of claim 11, wherein the pharmaceutical composition is administered a pharmaceutically actable route of administration selected from subcutaneous, intradermal, intravenous, intra-arterial, intraperitoneal, or intramuscular.

13. The method of claim 1, wherein the activin inhibitor is administered one or more times daily.

14. The method of claim 1, wherein the activin inhibitor is administered one or more times per week.

15. The method of claim 1, wherein the activin inhibitor is administered once per month.

16. The method of claim 9, wherein the pharmaceutical composition is administered one or more times daily.

17. The method of claim 9, wherein the pharmaceutical composition is administered one or more times per week.

18. The method of claim 9, wherein the pharmaceutical composition is administered once a month.

19. The method of claim 1, wherein the liver fibrosis in a subject is caused by one or more of disorders comprising autoimmune hepatitis, biliary obstruction, iron overload nonalcoholic fatty liver disease, which includes nonalcoholic fatty liver and nonalcoholic steatohepatitis, viral hepatitis B, hepatitis C, alcoholic liver disease or the long-term consumption of alcohol.

20. The method of claim 9, the liver fibrosis in a subject to be treated or prevented is caused by one or more of disorders comprising autoimmune hepatitis, biliary obstruction, iron overload nonalcoholic fatty liver disease, which includes nonalcoholic fatty liver and nonalcoholic steatohepatitis, viral hepatitis B, hepatitis C, alcoholic liver disease or the long-term consumption of alcohol.
